(19) United States
(12) Patent Application Publication
(10) Pub. No.: US 2010/0209396 A1
(43) Pub. Date: Aug. 19, 2010

(54) Method of Enhancing Proliferation and/or Hematopoietic Differentiation of Stem Cells

(75) Inventors: George Q. Daley, Weston, MA (US); Yuan Wang, Boston, MA (US); Frank Yates, Boston, MA (US); Olaia Naveiras, Cambridge, MA (US)

Correspondence Address: DAVID S. RESNICK, NIXON PEABODY LLP, 100 SUMMER STREET, BOSTON, MA, 02110-2131, US

(73) Assignee: CHILDREN'S MEDICAL CENTER CORPORATION, Boston, MA (US)

(21) Appl. No.: 11/993,310

(22) PCT Filed: Jun. 21, 2006
(86) PCT No.: PCT/US06/24099

§ 371 Date: Mar. 11, 2010

Related U.S. Application Data
(60) Provisional application No. 60/692,732, filed on Jun. 22, 2005.

(51) Int. Cl. C12N 5/0735; A61K 35/48

(52) U.S. Cl. 424/93.7; 435/377

(57) ABSTRACT

The present invention provides methods for inducing differentiation of a stem cell, such as an embryonic stem cell, into a hematopoietic stem cell, by expressing a cdx gene and/or a hox gene. The method is useful for generating expanded populations of hematopoietic stem cells (HSCs) and thus mature blood cell lineages. This is desirable where a mammal has suffered a decrease in hematopoietic or mature blood cells as a consequence of disease, radiation or chemotherapy.

### CROSS REFERENCE TO RELATED APPLICATIONS

This invention claims the benefit under 35 U.S.C. §119(e) of U.S. Provisional Patent Application Ser. No. 60/692,732 filed Jun. 22, 2005, the contents of which are herein incorporated by reference in their entirety.

### GOVERNMENT SUPPORT

This invention was supported by the National Institutes of Health—NIH/NHLBI Grant No. T32HL07623 and NIH/NIDDK Grant Nos. R01DK70055 and DK59279. The government of the United States has certain rights thereto.

### BACKGROUND OF THE INVENTION

Chemo- and radiation therapies cause dramatic reductions in blood cell populations in cancer patients. At least 500,000 cancer patients undergo chemotherapy and radiation therapy in the US and Europe each year and another 200,000 in Japan. Bone marrow transplantation therapy of value in aplastic anemia, primary immunodeficiency and acute leukemia (following total body irradiation) is becoming more widely practiced by the medical community. At least 15,000 Americans have bone marrow transplants each year. Other diseases can cause a reduction in entire or selected blood cell lineages. Examples of these conditions include anemia (including macrocytic and aplastic anemia); thrombocytopenia; hypoplasia; immune (autoimmune) thrombocytopenic purpura (ITP); and HIV induced ITP.

Pharmaceutical products are needed which are able to enhance reconstitution of blood cell populations of these patients.

### SUMMARY OF THE INVENTION

The present invention provides methods for inducing differentiation of a stem cell, such as an embryonic stem cell, into a hematopoietic stem cell, by adding cdx protein and/or hox protein. In one preferred embodiment, the cdx protein is added before the hox protein is added. In one embodiment, a cdx gene and/or a hox gene is expressed. In one preferred embodiment, the cdx gene is expressed before the hox gene is expressed. The method is useful for generating expanded populations of hematopoietic stem cells (HSCs) and thus mature blood cell lineages. This is desirable where a mammal has suffered a decrease in hematopoietic or mature blood cells as a consequence of disease, radiation or chemotherapy.

The method of the present invention comprises increasing the intracellular level of a cdx and a hox in stem cells, including embryonic stem (ES) cells and hematopoietic stem cells (HSCs), in culture, either by providing an exogenous cdx and/or an exogenous hox protein to the cell, or by introduction into the cell of a genetic construct encoding a cdx and/or a genetic construct encoding a hox. The cdx is selected from the cdx family and includes cdx1, cdx2, or cdx4. The cdx may be a wild type protein appropriate for the species from which the cells are derived, or a mutant form of the protein. The hox is selected from the hox family and includes hoxa4, hoxa6, hoxa7, hoxa9, hoxa10, hoxb1, hoxb3, hoxb4, hoxb5, hoxb6, hoxb7, hoxb8, hoxb9 or hoxc6. The hox may be a wild type protein appropriate for the species from which the cells are derived, or a mutant form of the protein.

In one embodiment, cdx4 protein and hoxb4 protein are added to embryonic stem cells, including, for example, human embryonic stem cells.

In one embodiment, cdx4 and hoxb4 are expressed in embryonic stem cells, including, for example, human embryonic stem cells.

In one embodiment, the cdx protein is added before the hox protein. In one preferred embodiment, the cdx protein can be added at least three days before the hox protein is added. In another embodiment, the cdx protein can be added at least one day before the hox protein is added.

In one embodiment, the cdx gene is expressed before the hox gene. In one preferred embodiment, the cdx gene can be expressed for at least three days before the hox gene is expressed. In another embodiment, the cdx gene can be expressed at least one day before the hox gene is expressed.

One embodiment of the invention provides a method for inducing differentiation of an embryonic stem cell into a hematopoietic stem cell, comprising introducing into the stem cells in an in vitro culture medium an exogenous protein comprising protein encoded by at least one gene selected from the group consisting of a cdx gene and a hox gene, and culturing the stem cells. In one embodiment, the exogenous protein is introduced via exogenous nucleic acid introduced into the stem cells and where each gene is operably linked to a promoter and the stem cells are cultured under conditions to express the gene(s) in the embryonic stem cell.

One embodiment of the invention provides a method for producing hematopoietic stem cells, by obtaining or generating a culture of embryonic stem cells, and introducing into the stem cells in an in vitro culture medium an exogenous protein comprising protein encoded by at least one gene selected from the group consisting of a cdx gene and a hox gene, and culturing the stem cells, thereby producing hematopoietic stem cells. In one embodiment, the exogenous protein is introduced via exogenous nucleic acid introduced into the stem cells and where each gene is operably linked to a promoter, and culturing the stem cells under conditions to express the gene(s) in the embryonic stem cell.

Another embodiment of the invention provides a method for enhancing proliferation or hematopoietic differentiation of a mammalian stem cell, by introducing into the stem cells in an in vitro culture medium an exogenous protein comprising protein encoded by at least one gene selected from the group consisting of a cdx gene and a hox gene, and culturing the stem cells, thereby enhancing proliferation or hematopoietic differentiation of a mammalian stem cell. In one embodiment, the exogenous protein is introduced via exogenous nucleic acid introduced into the stem cells and where each gene is operably linked to a promoter, and culturing the stem cells under conditions to express the gene(s) in the embryonic stem cell.

Any method for introducing protein into a stem cell can be used with the methods of the invention. In one embodiment, the exogenous protein is introduced into the cell by addition of the protein to the media in which the cultured. In one embodiment, the exogenous protein is introduced into the cell via cells in culture with the stem cells, wherein the cells in culture with the stem cells produce the exogenous protein.

Any method for introducing a gene into a stem cell can be used with the methods of the invention. In one embodiment, the exogenous nucleic acid is a retroviral vector. In another embodiment, the exogenous nucleic acid is an episomal vector.

The invention also provides methods of treating a mammal in need of improved hematopoietic capability, by introducing into a stem cell an exogenous protein comprising protein encoded by at least one gene selected from the group consisting of a cdx and a hox gene; culturing the stem cells under conditions to express the gene(s) in the stem cell, thereby enhancing proliferation or hematopoietic differentiation of the stem cells; and administering the cells to the mammal, thereby improving hematopoietic capability. In one embodiment, the exogenous protein is introduced via exogenous nucleic acid introduced into the stem cells and where each gene is operably linked to a promoter, and culturing the stem cells under conditions to express the gene(s) in the embryonic stem cell. In one embodiment, the stem cell is autologous. In one embodiment, the mammal is suffering from, or is susceptible to, decreased blood cell levels. Decreased blood cell levels can caused by chemotherapy, radiation therapy, bone marrow transplantation therapy, or congenital anemia.

The methods of the invention can be used with a variety of stern cells, including embryonic stem cells, umbilical cord blood stem cells, unrestricted somatic stem cells (USSC) derived from human umbilical cord blood, somatic stem cells, mesenchymal stem cells, mesenchymal progenitor cells, hematopoietic stem cells, hematopoietic lineage progenitor cells, endothelial stem cells, placental fetal stem cells, and endothelial progenitor cells.

In one embodiment, the stem cell is a mammalian stem cell, including murine stem cells and human stem cells.

### BRIEF DESCRIPTION OF THE DRAWINGS

FIGS. 1A-1B show hemangioblast colony forming and replating assays on day 3.2 EBs. FIG. 1A shows 3×104 EB cells after day 3.2 differentiation from an inducible Cdx4 cell line were plated in blast-colony forming media, in the absence or presence of doxycyclin (dox). Blast colonies were counted four days post plating. A photograph of one representative blast colony is shown. FIG. 1B shows individual colonies from the blast forming assay were picked (samples from different groups as indicated by arrows) and replating efficiency to form 2nd hematopoitic colonies was measured.

FIGS. 2A-2F show phenotypic analysis of ESC-derived hematopoietic progenitors from an inducible Cdx4 cell line. FIG. 2A shows day 6 EB cells were plated into methylcellulose (M3434) containing cytokines to support the growth of hematopoietic progenitor. Colonies were identified and counted from day 5 to 10 after plating. EryP: Primitive Erythroid Colonies. EryD: Definitive Erythroid Colonies. GEMM: Granulocyte, Erythroid, Macrophage, Megakaryocyte multilineage colony. GM: Granulocyte Macrophage myeloid colony. Mac: Macrophage colony. Mast: Mast cell colony. FIG. 2B shows relative expression levels of early and definitive hematopoietic genes in day 6 EBs by real-time RT-PCR analysis. FIG. 2C shows flow cytometry analysis of c-kit and CD41 on day 6 EBs. Samples from cells with ectopic Cdx4 expression induced by doxycyclin during day 3-6 of EB formation: +dox, day 3-6; non-induced: −dox. FIG. 2D shows ESC-derived cell expansion by Cdx4 activation on OP9 stromal cells. Inducible Cdx4 ESC were treated with doxycyclin from day 3-6 of EB formation and cultured on OP9 cells in the absence or presence of doxycyclin. Fold increase of cell number on day 18 of OP9 culture was calculated over that from the starting point. FIG. 2E shows relative expression levels of fetal hemoglobin (β-H1) and adult hemoglobin (β-major) before and after OP9 expansion by real-time RT-PCR analysis. FIG. 2F shows relative expression levels of genes specific to different hematopoietic and lymphoid development pathways in Cdx4-induced or HoxB4-induced ESC-derived hematopoietic progenitors, 15 days after OP9 expansion. Values in FIGS. 2B, 2E, 2F were obtained by real-time PCR and normalized against the expression of the (β-actin housekeeping gene.

FIGS. 3A-3G show donor cell chimerism and multilineage blood reconstitution in tissues of irradiated primary and secondary mice engrafted with ESC-derived hematopoietic stem cells, over time (weeks post trx). FIG. 3A shows schema for derivation of HSCs from ESCs. We engineered a tetracycline-inducible murine ESC line to conditionally express Cdx4 during EB differentiation. The expression of Cdx4 was induced by doxycycline during day 3 to 6 of EB development from ESCs, while a separate population of EBs was left uninduced. Day 6 EB cells from both groups were transduced with a retroviral vector expressing HoxB4 linked to GFP via IRES, and grown on OP9 stromal cells for 10-14 days. Cultured cells were then injected intravenously into lethally irradiated lymphocyte-deficient Rag2/γc double knockout mice. FIG. 3B shows donor chimerism (% GFP+ as defined by flow cytometry) in peripheral blood of mice engrafted with Cdx4, Hoxb4 or Cdx4/Hoxb4 modified hematopoietic populations differentiated from embryonic stem cells at 8 weeks post transplantation; FIG. 3C shows donor chimerism (% GFP+ as defined by flow cytometry) in peripheral blood of mice engrafted with Hoxb4 or Cdx4/Hoxb4 modified hematopoietic populations differentiated from embryonic stem cells over 22 weeks post transplantation; FIG. 3D shows flow cytometry analysis of peripheral blood cells expressing either myeloid antigens (Gr-1; M) or lymphoid antigens (CD3/B220; L). The numbers in boxes represent the number of mice analyzed at that time point. FIG. 3E shows donor chimerism in peripheral blood of secondary animals. Bone marrow (BM) from primary recipients after at least 12 weeks post transplantation were sorted and transplanted into secondary recipients. FIG. 3F shows myeloid-lymphoid reconstitution of splenocytes from secondary animals. FIG. 3G shows flow cytometric analysis of bone marrow (BM) and spleen cells in long-term engrafted animals (7 months) with ESC-derived HSCs, showing donor cell reconstitution of myeloid, erythroid, B and T lineages. Rv-Hoxb4: ESCs infected with HoxB4 retrovirus alone; icdx4/Rv-HoxB4: ESCs modified with cdx4 induction followed by retroviral transduction with HoxB4; GFP: mice engrafted with BM carrying a GFP transgene driven by the chicken γ-actin promoter (Okabe, 1997); Rag2-γc: lymphocyte-deficient recipient mice. The numbers in each panel indicate the percentage of positively stained cells. Given that recipient mice are genetically lymphoid-deficient (Colucci, 1999), all lymphoid cells are donor-derived. See FIG. 7 for data on retroviral silencing. The error bars in each panel represent standard deviation. 1ry: primary; 2ry: secondary.

FIGS. 4A-4C show clonal analysis of hematopoietic populations of mice engrafted with ESC-derived HSCs, as determined by Southern hybridization analysis of retroviral integration sites. FIG. 4A shows structure of the retroviral vector MSCV-HoxB4-ires-GFP. Probes used in Southern hybridization analysis are indicated. FIG. 5B on the left shows southern analysis of fractionated myeloid and lymphoid populations from two primary (1ry) and one secondary (2ry) engrafted mice, showing multiple co-migrating fragments. B/G: Gr-1+ myeloid cells from bone marrow; S/L: CD3+/B220+ lymphoid cells from Spleen; FIG. 4B on the right shows bone marrow and spleen cells from two primary engrafted animals and comparable tissue from the corresponding secondary animals, showing co-migrating fragments. FIG. 4C shows Southern analysis of hematopoietic tissues from one primary and two corresponding secondary recipients engrafted with ESC-HSCs: spleen (S), BM (B), Gr1+ BM cells (B/G), Gr1+ splenocytes (S/G), and CD3+ or B220+ splenic lymphocytes (S/L). Mye/Lym represents the ratio of Gr-1+ cells to CD3+ and B220+ populations in corresponding sample, as determined by flow cytometry. Bone marrow consisted primarily of myeloid cells, while spleen was a mixed population. Relative DNA level was calculated by comparing endogenous HoxB4 (endog) with control (DNA isolated from Ainv15 ES cells). Proviral copy number was calculated by comparing the level of proviral HoxB4 (Rv-HoxB4) with endogenous HoxB4 level. Weeks post-transplantation (trx) are indicated under the figure. All samples were taken from mice engrafted with Cdx4/HoxB4 treated cells, except the 3rd and 4th lanes in FIG. 3B, left panel, which were harvested from a mouse transplanted with HoxB4 treated cells.

FIG. 5A-5D shows flow cytometric analysis of hematopoietic multi-lineage contribution in engrafted mice transplanted with ESC-derived HSCs. FIGS. 5A-5B show flow cytometric analysis of lymphoid populations isolated from spleen (FIG. 5A), thymus (FIG. 5A) and lymph (FIG. 5B) nodes of the primary recipients (1ry) 7 months post transplantation. FIGS. 5C-5D show flow cytometric analysis of lymphoid, myeloid, and erythroid (Ter119) populations isolated from spleen (FIG. 5C), thymus (FIG. 5D) and BM (FIG. 5D) of a representative secondary recipient (2ry) 4 months post transplantation. The numbers in each panel indicate the percentage of positively stained cells.

FIG. 6 shows post-sorting analysis. Flow cytometric analysis on Gr-1 sorted BM cells or B220/CD3 sorted splenocytes from a representative secondary recipient used in Southern Blot analysis, showing high purity (FIG. 6). In both cases, sorted cells were stained with PE-conjugated anti-rat IgG antibody.

FIGS. 7A-7B show retroviral silencing in infected hematopoietic stem cells. FIG. 7A shows flow cytometric analysis of engrafted lymphoid cells (B220, CD3) shows a predominantly GFP negative population in the spleen from a primary mouse 7 months post transplantation. Because recipient animals are lymphoid deficient, these data suggest transcriptional silencing of the integrated retrovirus in donor cells. This is also implied by our proviral copy number data, which showed between 1-3 copies/cell in most animals (FIG. 2). FIG. 7B shows GFP-negative and positive B220+ and CD3+ splenocytes were isolated by FACS, and genomic DNA was subjected to quantitative real time PCR amplification of proviral sequences (GFP). These data show equivalent levels of proviral DNA in both GFP positive and negative cells, thereby establishing the presence of transcriptionally inactive provirus in the GFP-negative cells (Klug, 2000). Transcriptional silencing thus accounts in part for incomplete donor chimerism of engrafted mice, when assayed by total GFP content in hematopoietic tissues (FIG. 3). GFP DNA levels are expressed in arbitrary units using the comparative CT method (relative to the TDAG51 gene as an internal normalization control).

FIG. 8 shows the results of Cdx4 expression during in vitro ES differentiation. FIG. 8 shows quantification of the results of RT-PCR/Northern blot analysis of Cdx4 expression during embryoid body (EB) development as relative expression level during different days.

FIG. 9 shows that ectopic Cdx4 expression induces Hox gene expression in hematopoietic cells, including HoxA1, HoxA2, HoxA4, HoxA6, HoxA7, HoxA9, HoxA10, HoxB1, HoxB2, HoxB3, HoxB4, HoxB6, HoxB7, HoxB8, HoxB9, and HoxC6. Hematopoietic cells which are analyzed include Flk1−, day 4 EBs, Flk1+, day 4 EBs, and CD41+ day 6 EBs.

FIG. 10 shows the results of TAT-HA-Hoxb4 protein transduction on EBs. TAT-HA-HoxB4 protein was transduced every 3 hours for a total of 12 hours (5 administrations). The results of a Methocult 3434 assay are shown.

### DETAILED DESCRIPTION OF THE INVENTION

The present invention provides methods for inducing differentiation of a stem cell, such as an embryonic stem cell, into a hematopoietic stem cell, by adding cdx protein and/or hox protein. In one embodiment, cdx protein and/or hox protein is added by expressing a cdx gene and/or a hox gene. The method is useful for generating expanded populations of hematopoietic stem cells (HSCs) and thus mature blood cell lineages. This is desirable where a mammal has suffered a decrease in hematopoietic or mature blood cells as a consequence of disease, radiation, chemotherapy or congenital anemia (e.g., Diamond Blackfan Anemia). The expanded populations of HSCs generated by the methods of the present invention are useful for transplanting into a subject in need thereof. Thus, the HSCs may repopulate or reconstitute hematopoietic lineages in the subject.

The method of the present invention comprises adding exogenous protein encoded by cdx and/or hox genes to stem cells. The exogenous protein can be added by expressing cdx and/or hox in stem cells. The cdx is selected from the cdx family and includes cdx1, cdx2, or cdx4. The cdx may be a wild type protein appropriate for the species from which the cells are derived, or a mutant form of the protein. The hox is selected from the hox family and includes hoxa4, hoxa6, hoxa7, hoxa9, hoxa10, hoxb1, hoxb3, hoxb4, hoxb5, hoxb6, hoxb7, hoxb8, hoxb9 or hoxc6. The hox may be a wild type protein appropriate for the species from which the cells are derived, or a mutant form of the protein.

Protein encoded by a cdx and a hox gene can both be added to a stem cell. In one embodiment, the protein is added by expressing both a cdx and a hox. In certain preferred methods, the protein encoded by the cdx gene can be added before the protein encoded by the hox gene. In one embodiment, the protein encoded by the cdx gene is added before the protein encoded by the hox gene. In one embodiment, the protein encoded by the cdx gene is added at least about half a day, about 1 day, about 2 days, about 3 days, about 4 days, about 5 days, about 6 days, about 7 days, about 8 days, about 9 days, about 10 days, about 11 days, about 12 days, about 13 days, about 14 days before the protein encoded by the hox gene is added. In one preferred embodiment, the protein encoded by the cdx gene is added at least three days before the protein encoded by the hox gene is added. In another preferred embodiment, the protein encoded by the cdx gene is added at least one day before the protein encoded by the hox gene is added.

In one preferred method of the invention, protein encoded by cdx4 and hoxb4 is added sequentially in embryonic stem cells.

In one embodiment, mammalian stem cells are differentiated to HSCs in vitro by increasing the level of cdx and hox in the cell. In another embodiment, the number of HSCs in a culture is expanded by increasing the levels of cdx and hox in the cell. The intracellular levels of cdx and hox may be manipulated by providing exogenous cdx and/or hox protein to the cell, or by introduction into the cell of a genetic construct encoding a cdx and/or a hox. The cdx and/or the hox may be a wild-type or a mutant form of the protein.

In one embodiment, the exogenous protein is added to the cell by cells in the same culture as the target cell. For example, cells in the same culture as the stem cell can be feeder cells, stroma cells or other supporting cells. The exogenous protein can be synthesized by the supporting cells and thus introduced to the stem cell. For example, the exogenous protein can be expressed and secreted into the medium. The exogenous protein may be expressed from a vector or recombinant expression cassette introduced into the cells in culture with the stem cell. See for example, Nature Medicine, 2003, 9, 1423-1427 and 1428-1432.

The term cdx, as used herein, is intended to refer to both wild-type and mutant forms of the cdx protein family, and to fusion proteins and derivatives thereof. Usually the protein will be of mammalian origin, although the protein from other species may find use. The sequences of many cdx proteins are publicly known. Preferably, the mammal is a human and the cdx is selected from the group consisting cdx1 (GenBank accession number NM_{—}001804 (human), NM_{—}009880 (mouse); Suh et al., J. Biol. Chem. 277:35795 (2002)), cdx2 (GenBank accession number NM_{—}001265 (human), NM_{—}007673 (mouse); Yamamoto et al., Biochem. Biophys. Res. Commun. 300(4):813 (2003)), and cdx4 (GenBank accession number NM 005193 (human), NM_{—}7674 (mouse); Horn et al., Hum. Mol. Genet. 4(6), 1041-1047 (1995)).

The term hox, as used herein, is intended to refer to both wild-type and mutant forms of the hox protein family, and to fusion proteins and derivatives thereof. Usually the protein will be of mammalian origin, although the protein from other species may find use. The sequences of many hox proteins are publicly known. Preferably, the mammal is a human and the hox is selected from the group consisting of) hoxa4 (GenBank accession numbers NM_{—}002141 (human); NM_{—}008265 (mouse)), hoxa6 (GenBank accession numbers NM_{—}024014 (human); NM_{—}010454 (mouse)), hoxa7 (GenBank accession numbers NM_{—}006896 (human); NM_{—}010455 (mouse)), hoxa9 (GenBank accession numbers NM_{—}152739, NM_{—}002142 (human); NM_{—}010456 (mouse)), hoxa10 (GenBank accession numbers NM_{—}153715, NM_{—}018951 (human); NM_{—}008263 (mouse)), hoxb1 (GenBank accession numbers NM_{—}002144 (human); NM_{—}008266 (mouse)), hoxb3 (GenBank accession numbers NM_{—}002146 (human); NM_{—}010458 (mouse)), hoxb4 (GenBank accession numbers NM_{—}024015 (human); NM_{—}010459 (mouse)), hoxb5 (GenBank accession numbers NM_{—}002147 (human); NM_{—}008268 (mouse)), hoxb6 (GenBank accession numbers NM_{—}156037, NM_{—}018952 NM_{—}156036 (human); NM_{—}008269 (mouse)), hoxb7 (GenBank accession numbers NM_{—}004502 (human); NM_{—}010460 (mouse)), hoxb8 (GenBank accession numbers NM_{—}024016 (human); NM_{—}010461 (mouse)), hoxb9 (GenBank accession numbers NM_{—}024017 (human); NM_{—}008270 (mouse)) or hoxc6 (GenBank accession numbers NM_{—}004503 (human); NM_{—}010465 (mouse)).

One embodiment of the invention provides a method for inducing differentiation of an embryonic stem cell into a hematopoietic stem cell, comprising introducing into said stem cells in an in vitro culture medium an exogenous protein comprising at least one protein encoded by a gene selected from the group consisting of a cdx gene and a hox gene, and culturing said stem cells, thereby inducing its differentiation into a hematopoietic stem cell. In one embodiment, the exogenous protein is introduced via introduction of nucleic acid into the stem cells, wherein each gene is operably linked to a promoter, and said stem cells are cultured under conditions to express said gene(s) in the embryonic stem cell.

One embodiment of the invention provides a method for producing hematopoietic stem cells, by obtaining or generating a culture of embryonic stem cells, and introducing into the stem cells in an in vitro culture medium an exogenous protein comprising protein encoded by at least one gene selected from the group consisting of a cdx gene and a hox gene, and culturing the stem cells, thereby producing hematopoietic stem cells. In one embodiment, the exogenous protein is introduced via introduction of nucleic acid into the stem cells, wherein each gene is operably linked to a promoter, and said stem cells are cultured under conditions to express said gene(s) in the embryonic stem cell.

Another embodiment of the invention provides a method for enhancing proliferation or hematopoietic differentiation of a mammalian stem cell, by introducing into the stem cells in an in vitro culture medium an exogenous protein comprising protein encoded by at least one gene selected from the group consisting of a cdx gene and a hox gene, and culturing the stem cells, thereby enhancing proliferation or hematopoietic differentiation of a mammalian stem cell. In one embodiment, the exogenous protein is introduced via introduction of nucleic acid into the stem cells, wherein each gene is operably linked to a promoter, and said stem cells are cultured under conditions to express said gene(s) in the embryonic stem cell.

The differentiated and expanded cell populations are useful as a source of hematopoietic stem cells, which may be used in transplantation to restore hematopoietic function to autologous or allogeneic recipients.

Any method for introducing a gene into a stem cell can be used with the methods of the invention. In one embodiment, the exogenous nucleic acid is a retroviral vector. In another embodiment, the exogenous nucleic acid is an episomal vector.

The invention also provides methods of treating a mammal in need of improved hematopoietic capability, by introducing into a stem cell an exogenous protein comprising protein encoded by at least one gene selected from the group consisting of a cdx and a hox gene; culturing the stem cells, thereby enhancing proliferation or hematopoietic differentiation of the stem cells; and administering the cells to the mammal, thereby improving hematopoietic capability. In one embodiment, the exogenous protein is introduced via introduction of nucleic acid into the stem cells, wherein each gene is operably linked to a promoter, and said stem cells are cultured under conditions to express said gene(s) in the embryonic stem cell. In one embodiment, the stem cell is autologous. In one embodiment, the mammal is suffering from, or is susceptible to, decreased blood cell levels. Decreased blood cell levels can be caused by chemotherapy, radiation therapy, bone marrow transplantation therapy, or congenital anemia.

Stem cells are undifferentiated cells defined by their ability at the single cell level to both self-renew and differentiate to produce progeny cells, including self-renewing progenitors, non-renewing progenitors and terminally differentiated cells. Stem cells are also characterized by their ability to differentiate in vitro into functional cells of various cell lineages from multiple germ layers (endoderm, mesoderm and ectoderm), as well as to give rise to tissues of multiple germ layers following transplantation and to contribute substantially to most, if not all, tissues following injection into blastocysts.

Stem cells are classified by their developmental potential as: (1) totipotent—able to give rise to all embryonic and extraembryonic cell types; (2) pluripotent—able to give rise to all embryonic cell types; (3) multipotent—able to give rise to a subset of cell lineages, but all within a particular tissue, organ, or physiological system (for example, hematopoietic stem cells (HSC) can produce progeny that include HSC (self-renewal), blood cell-restricted oligopotent progenitors, and all cell types and elements (e.g., platelets) that are normal components of the blood); (4) oligopotent—able to give rise to a more restricted subset of cell lineages than multipotent stem cells; and (5) unipotent—able to give rise to a single cell lineage (e.g., spermatogenic stem cells).

Stem cells are also categorized on the basis of the source from which they may be obtained. An embryonic stem cell is a pluripotent cell from the inner cell mass of a blastocyst-stage embryo. A fetal stem cell is one that originates from fetal tissues or membranes. A postpartum stem cell is a multipotent or pluripotent cell that originates substantially from extraembryonic tissue available after birth, namely, the placenta and the umbilical cord. These cells have been found to possess features characteristic of pluripotent stem cells, including rapid proliferation and the potential for differentiation into many cell lineages. Postpartum stem cells may be blood-derived (e.g., as are those obtained from umbilical cord blood) or non-blood-derived (e.g., as obtained from the non-blood tissues of the umbilical cord and placenta). An adult stem cell is generally a multipotent undifferentiated cell found in tissue comprising multiple differentiated cell types. The adult stem cell can renew itself and, under normal circumstances, differentiate to yield the specialized cell types of the tissue from which it originated, and possibly other tissue types.

Embryonic tissue is typically defined as tissue originating from the embryo (which in humans refers to the period from fertilization to about six weeks of development. Fetal tissue refers to tissue originating from the fetus, which in humans refers to the period from about six weeks of development to parturition. Extraembryonic tissue is tissue associated with, but not originating from, the embryo or fetus. Extraembryonic tissues include extraembryonic membranes (chorion, amnion, yolk sac and allantois), umbilical cord and placenta (which itself forms from the chorion and the maternal decidua basalis).

Differentiation is the process by which an unspecialized (“uncommitted”) or less specialized cell acquires the features of a specialized cell, such as a nerve cell or a muscle cell, for example. A differentiated or differentiation-induced cell is one that has taken on a more specialized (“committed”) position within the lineage of a cell. The term committed, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. De-differentiation refers to the process by which a cell reverts to a less specialized (or committed) position within the lineage of a cell. As used herein, the lineage of a cell defines the heredity of the cell, i.e., which cells it came from and what cells it can give rise to. The lineage of a cell places the cell within a hereditary scheme of development and differentiation. A lineage-specific marker refers to a characteristic specifically associated with the phenotype of cells of a lineage of interest and can be used to assess the differentiation of an uncommitted cell to the lineage of interest.

In a broad sense, as used herein, a progenitor cell is a cell that has the capacity to create progeny that are more differentiated than itself and yet retains the capacity to replenish the pool of progenitors. By that definition, stem cells themselves are also progenitor cells, as are the more immediate precursors to terminally differentiated cells. When referring to the cells of the present invention, as described in greater detail below, this broad definition of progenitor cell may be used. In a narrower sense, a progenitor cell is often defined as a cell that is intermediate in the differentiation pathway, i.e., it arises from a stem cell and is intermediate in the production of a mature cell type or subset of cell types. This type of progenitor cell is generally not able to self-renew. Accordingly, if this type of cell is referred to herein, it will be referred to as a non-renewing progenitor cell or as an intermediate progenitor or precursor cell.

The term stem cell is used herein to refer to a mammalian cell that has the ability both to self-renew, and to generate differentiated progeny (see Morrison et al. (1997) Cell 88:287-298). Generally, stem cells also have one or more of the following properties: an ability to undergo asynchronous, or asymmetric replication, that is where the two daughter cells after division can have different phenotypes; extensive self-renewal capacity; capacity for existence in a mitotically quiescent form; and clonal regeneration of all the tissue in which they exist, for example the ability of hematopoietic stem cells to reconstitute all hematopoietic lineages. “Progenitor cells” differ from stem cells in that they typically do not have the extensive self-renewal capacity, and often can only regenerate a subset of the lineages in the tissue from which they derive, for example only lymphoid, or erythroid lineages in a hematopoietic setting.

Stem cells may be characterized by both the presence of markers associated with specific epitopes identified by antibodies and the absence of certain markers as identified by the lack of binding of specific antibodies. Stem cells may also be identified by functional assays both in vitro and in vivo, particularly assays relating to the ability of stem cells to give rise to multiple differentiated progeny.

In one preferred embodiment, the stem cell is an embryonic stem cell. Embryonic stem cells, sometimes referred to as ES cells or ESCs, are cultured cells derived from the pluripotent inner cell mass of blastocyst stage embryos, that are capable of replicating indefinitely. In general, ES cells have the potential to differentiate into other cells (i.e., they are pluripotent); thus, they may serve as a continuous source of new cells. By “blastocyst” is meant the mammalian conceptus in the post-morula stage, consisting of the trophoblast and an inner cell mass. An “ES cell clone” as used herein is a subpopulation of cells derived from a single cell of the ES cell population following introduction of DNA and subsequent selection. The embryonic stem cell of the present invention may be obtained from any animal, but is preferably obtained from a mammal (e.g., human, domestic animal, or commercial animal). In one embodiment of the present invention, the embryonic stem cell is a murine embryonic stem cell. In another, preferred, embodiment, the embryonic stem cell is obtained from a human.

Other preferred stem cells include somatic stem cells, umbilical cord blood stem cells, unrestricted somatic stem cells (USSC) derived from human umbilical cord blood, placenta-derived stem cells, postpartum-derived cells, mesenchymal stem cells, mesenchymal progenitor cells, hematopoietic lineage stem cells, hematopoietic lineage progenitor cells, endothelial stem cells, placental fetal stem cells, and endothelial progenitor cells.

In one aspect, the invention provides postpartum-derived cells (PPDCs) derived from postpartum tissue substantially free of blood. The PPDCs may be derived from placenta of a mammal including but not limited to human. The cells are capable of self-renewal and expansion in culture. The postpartum-derived cells have the potential to differentiate into cells of other phenotypes. The invention provides, in one of its several aspects cells that are derived from umbilical cord, as opposed to umbilical cord blood. The invention also provides, in one of its several aspects, cells that are derived from placental tissue. Subsets of the cells of the present invention are referred to as placenta-derived cells (PDCs) or umbilical cord-derived cells (UDCs). PPDCs of the invention encompass undifferentiated and differentiation-induced cells. In addition, the cells may be described as being stem or progenitor cells, the latter term being used in the broad sense. The term derived is used to indicate that the cells have been obtained from their biological source and grown or otherwise manipulated in vitro (e.g., cultured in a growth medium to expand the population and/or to produce a cell line).

Somatic tissue stem cells of the present invention can include any stem cells isolated from adult tissue. Somatic stem cells include but are not limited to bone marrow derived stem cells, adipose derived stem cells, and mesenchymal stem cells. Bone marrow derived stem cells refers to all stem cells derived from bone marrow; these include but are not limited to mesenchymal stem cells, bone marrow stromal cells, and hematopoietic stem cells. Bone marrow stem cells are also known as mesenchymal stem cells or bone marrow stromal stem cells, or simply stromal cells or stem cells. In one embodiment, the bone marrow stems are circulating bone marrow stem cells.

In some embodiments, somatic tissue stem cells can be isolated from fresh bone marrow or adipose tissue by fractionation using fluorescence activated call sorting (FACS) with unique cell surface antigens to isolate specific subtypes of stem cells (such as bone marrow or adipose derived stem cells) for injection into recipients following expansion in vitro, as described above.

As stated above, stem cells can be derived from the individual to be treated or a matched donor. Those having ordinary skill in the art can readily identify matched donors using standard techniques and criteria. Cells can be obtained from donor tissue by dissociation of individual cells from the connecting extracellular matrix of the tissue. Tissue is removed using a sterile procedure, and the cells are dissociated using any method known in the art including treatment with enzymes such as trypsin, collagenase, and the like, or by using physical methods of dissociation such as with a blunt instrument.

The present invention provides a method for inducing differentiation of a stem cell, including an embryonic stem cell, into a differentiated hematopoietic stem cell, and a differentiated hematopoietic stein cell produced by this method. As used herein, the term “inducing differentiation of an embryonic stem cell” means activating, initiating, or stimulating a stem cell to undergo differentiation—the cellular process by which cells become structurally and functionally specialized during development.

As further used herein, a “differentiated hematopoietic stem cell” is a partially-differentiated or fully-differentiated hematopoietic stem cell, sometimes referred to simply as a hematopoietic stem cell or a HSC. HSCs typically have long-term engrafting potential in vivo. Animal models for long-term engrafting potential of candidate human hematopoietic stem cell populations include the SCID-hu bone model (Kyoizumi et al., Blood 79:1704 (1992); Murray et al., Blood 85 368-378 (1995)) and the in utero sheep model (Zanjani et al., J. Clin. Invest. 89:1179 (1992)). For a review of animal models of human hematopoiesis, see Srour et al., J. Hematother. 1:143-153 (1992) and the references cited therein. At present, a preferred in vitro assay for stem cells is the long-term culture-initiating cell (LTCIC) assay, based on a limiting dilution analysis of the number of clonogenic cells produced in a stromal co-culture after 5-8 weeks (Sutherland et al., Proc. Nat'l Acad. Sci. 87:3584-3588 (1990)). The LTCIC assay has been shown to correlate with another commonly used stem cell assay, the cobblestone area forming cell (CAFC) assay, and with long-term engrafting potential in vivo (Breems et al., Leukemia 8:1095 (1994)).

The cells of interest are typically mammalian, where the term refers to any animal classified as a mammal, including humans, domestic and farm animals, and zoo, laboratory, sports, or pet animals; such as dogs, horses, cats, cows, mice, rats, rabbits, etc. Preferably, the mammal is human.

The cells which are employed may be fresh, frozen, or have been subject to prior culture. They may be fetal, neonate, adult. Hematopoietic cells may be obtained from fetal liver, bone marrow, blood, particularly G-CSF or GM-CSF mobilized peripheral blood, cord blood or any other conventional source. The manner in which the stem cells are separated from other cells is not critical to this invention. As described above, a substantially homogeneous population of stem or progenitor cells may be obtained by selective isolation of cells free of markers associated with differentiated cells, while displaying epitopic characteristics associated with the stem cells.

The stem or progenitor cells are grown in vitro in an appropriate liquid nutrient medium. Generally, the seeding level will be at least about 10 cells/ml, more usually at least about 100 cells/ml and generally not more than about 105 cells/ml, usually not more than about 104 cells/ml.

Various media are commercially available and may be used, including Ex vivo serum free medium; Dulbecco's Modified Eagle Medium (DMEM), RPMI, Iscove's medium, etc. The medium may be supplemented with serum or with defined additives. Appropriate antibiotics to prevent bacterial growth and other additives, such as pyruvate (0.1-5 mM), glutamine (0.5-5 mM), 2-mercaptoethanol may also be included.

Culture in serum-free medium is of particular interest. The medium may be any conventional culture medium, generally supplemented with additives such as iron-saturated transferrin, human serum albumin, soy bean lipids, linoleic acid, cholesterol, alpha thioglycerol, crystalline bovine hemin, etc., that allow for the growth of hematopoietic cells.

Preferably the expansion medium is free of cytokines, particularly cytokines that induce cellular differentiation. The term cytokine may include lymphokines, monokines and growth factors. Included among the cytokines are thrombopoietin (TPO); nerve growth factors; platelet-growth factor; transforming growth factors (TGFs); erythropoietin (EPO); interferons such as interferon-α, β, and γ; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1γ, IL-2, IL-3, IL-4, IL-5, IL-6, IL-7, IL-8, IL-9, IL-11, IL-12; etc. In some circumstances, proliferative factors that do not induce cellular differentiation may be included in the cultures, e.g. c-kit ligand, LIF, and the like.

Frequently stem cells are isolated from biological sources in a quiescent state. Certain expression vectors, particularly retroviral vectors, do not effectively infect non-cycling cells. Cultures established with these vectors as a source of cdx sequences are induced to enter the cell cycle by a short period of time in culture with growth factors. For example, hematopoietic stem cells are induced to divide by culture with c-kit ligand, which may be combined with LIF, IL-11 and thrombopoietin. After 24 to 72 hours in culture with cytokines, the medium is changed, and the cells are exposed to the retroviral culture, using culture conditions as described above.

After seeding the culture medium, the culture medium is maintained under conventional conditions for growth of mammalian cells, generally about 37° C. and 5% CO2 in 100% humidified atmosphere. Fresh media may be conveniently replaced, in part, by removing a portion of the media and replacing it with fresh media. Various commercially available systems have been developed for the growth of mammalian cells to, provide for removal of adverse metabolic products, replenishment of nutrients, and maintenance of oxygen. By employing these systems, the medium may be maintained as a continuous medium, so that the concentrations of the various ingredients are maintained relatively constant or within a predescribed range. Such systems can provide for enhanced maintenance and growth of the subject cells using the designated media and additives.

The cdx and hox genes can be delivered to the stem cells by any means known in the art. In one embodiment of the invention, the cdx and hox are delivered to the targeted stem cells by introduction of an exogenous nucleic acid expression vector into the cells. Many vectors useful for transferring exogenous genes into target mammalian cells are available. The vectors may be episomal, e.g. plasmids, virus derived vectors such cytomegalovirus, adenovirus, etc., or may be integrated into the target cell genome, through homologous recombination or random integration, e.g. retrovirus derived vectors such MMLV, HIV-1, ALV, etc.

Retrovirus based vectors have been shown to be particularly useful when the target cells are hematopoietic stem cells. For example, see Baum et al. (1996) J Hematother 5(4):323-9; Schwarzenberger et al. (1996) Blood 87:472-478; Nolta et al. (1996) P.N.A.S. 93:2414-2419; and Maze et al. (1996) P.N.A.S. 93:206-210. Lentivirus vectors have also been described for use with hematopoietic stem cells, for example see Mochizuki et al. (1998) J Virol 72(11):8873-83. The use of adenovirus based vectors with hematopoietic cells has also been published, see Ogniben and Haas (1998) Recent Results Cancer Res 144:86-92.

Various-techniques known in the art may be used to transfect the target cells, e.g. electroporation, calcium precipitated DNA, fusion, transfection, lipofection and the like. The particular manner in which the DNA is introduced is not critical to the practice of the invention.

Combinations of retroviruses and an appropriate packaging line may be used, where the capsid proteins will be functional for infecting the target cells. Usually, the cells and virus will be incubated for at least about 24 hours in the culture medium. Commonly used retroviral vectors are “defective”, i.e. unable to produce viral proteins required for productive infection. Replication of the vector requires growth in the packaging cell line.

The host cell specificity of the retrovirus is determined by the envelope protein, env (p120). The envelope protein is provided by the packaging cell line. Envelope proteins are of at least three types, ecotropic, amphotropic and xenotropic. Retroviruses packaged with ecotropic envelope protein, e.g. MMLV, are capable of infecting most murine and rat cell types. Ecotropic packaging cell lines include BOSC23 (Pear et al. (1993) P.N.A.S. 90:8392-8396). Retroviruses bearing amphotropic envelope protein, e.g. 4070A (Danos et al, supra.), are capable of infecting most mammalian cell types, including human, dog and mouse. Amphotropic packaging cell lines include PA12 (Miller et al. (1985) Mol. Cell. Biol. 5:431-437); PA317 (Miller et al. (1986) Mol. Cell. Biol. 6:2895-2902) GRIP (Danos et al. (1988) PNAS 85:6460-6464). Retroviruses packaged with xenotropic envelope protein, e.g. AKR env, are capable of infecting most mammalian cell types, except murine cells.

The sequences at the 5′ and 3′ termini of the retrovirus are long terminal repeats (LTR). A number of LTR sequences are known in the art and may be used, including the MMLV-LTR; HIV-LTR; AKR-LTR; FIV-LTR; ALV-LTR; etc. Specific sequences may be accessed through public databases. Various modifications of the native LTR sequences are also known. The 5′ LTR acts as a strong promoter, driving transcription of the cdx gene after integration into a target cell genome. For some uses, however, it is desirable to have a regulatable promoter driving expression. Where such a promoter is included, the promoter function of the LTR will be inactivated. This is accomplished by a deletion of the U3 region in the 3′ LTR, including the enhancer repeats and promoter, that is sufficient to inactivate the promoter function. After integration into a target cell genome, there is a rearrangement of the 5′ and 3′ LTR, resulting in a transcriptionally defective provirus, termed a “self-inactivating vector”.

Suitable inducible or conditional promoters are activated in a desired target cell type, either the transfected cell, or progeny thereof. Alternatively, environmental factors or exogenous signals (e.g., transactivators) may be used to activate the inducible or conditional promoter. In one embodiment, the cdx gene(s) and the hox gene(s) are each under the control of different inducible promoters. By transcriptional activation, it is intended that transcription will be increased above basal levels in the target cell by at least about 100 fold, more usually by at least about 1000 fold. Various promoters are known that are induced in hematopoietic cell types, e.g. IL-2 promoter in T cells, immunoglobulin promoter in B cells, etc.

In one embodiment, the exogenous protein, e.g., cdx protein, e.g., hox protein, is introduced into the stem cell via cells in culture with the stem cells, i.e., co-cultured with the stem cells. The cells in culture with the stem cells produce the exogenous protein. The exogenous protein may be secreted into the media and thus introduced into the stem cell. The cells in culture with the stem cells may comprise a “feeder layer” of cells. The cells in culture with the stem cells may be transgenic for expression constructs directing the expression of the exogenous protein. Inducible promoters may direct the expression of the exogenous protein in the cells in culture with the stem cells.

In an alternative method, expression vectors that provide for the transient expression in mammalian cells may be used. In general, transient expression involves the use of an expression vector that is able to replicate efficiently in a host cell, such that the host cell accumulates many copies of the expression vector and, in turn, synthesizes high levels of a desired polypeptide encoded by the expression vector. Transient expression systems, comprising a suitable expression vector and a host cell, allow for the convenient short term expansion of cells, but do not affect the long term genotype of the cell.

In some cases it may be desirable to provide exogenous cdx protein and/or exogenous hox protein, rather than transducing the cells with an expression construct. The cdx protein and/or the hox protein may be added to the culture medium at high levels. Preferably the cdx and/or hox proteins are modified so as to increase transport into the cells. See, for example, US 2002/0086383. Preferably the cdx and/or hox proteins are modified so as to modulate protein turnover in the cell.

Any peptide, e.g., basic peptide, or fragment thereof, which is capable of crossing a biological membrane, either in vivo or in vitro, is included in the invention. These peptides can be synthesized by methods known to one of skill in the art. For example, several peptides have been identified which may be used as carrier peptides in a fusion protein in the methods of the invention for transducing proteins across biological membranes. These peptides include, for example, the homeodomain of antennapedia, a Drosophila transcription factor (Wang et al., (1995) PNAS USA., 92, 3318-3322); a fragment representing the hydrophobic region of the signal sequence of Kaposi fibroblast growth factor with or without NLS domain (Antopolsky et al. (1999) Bioconj. Chem., 10, 598-606); a signal peptide sequence of Caiman crocodylus Ig(5) light chain (Chaloin et al. (1997) Biochem. Biophys. Res. Comm., 243, 601-608); a fusion sequence of HIV envelope glycoprotein gp4114, (Morris et al. (1997) Nucleic Acids Res., 25, 2730-2736); a transportan A-achimeric 27-mer consisting of N-terminal fragment of neuropeptide galanine and membrane interacting wasp venom peptide mastoporan (Lindgren et al., (2000), Bioconjugate Chem., 11, 619-626); a peptide derived from influenza virus hemagglutinin envelop glycoprotein (Bongartz et al., 1994, Nucleic Acids Res., 22, 468 1 4688); RGD peptide; and a peptide derived from the human immunodeficiency virus type-1 (“HIV-1”). Purified HIV-1 TAT protein is taken up from the surrounding medium by human cells growing in culture (A. D. Frankel and C. O. Pabo, (1988) Cell, 55, pp. 1189-93). TAT protein trans-activates certain HIV genes and is essential for viral replication. The full-length HIV-1 TAT protein has 86 amino acid residues. The HIV tat gene has two exons. TAT amino acids 1-72 are encoded by exon 1, and amino acids 73-86 are encoded by exon 2. The full-length TAT protein is characterized by a basic region which contains two lysines and six arginines (amino acids 47-57) and a cysteine-rich region which contains seven cysteine residues (amino acids 22-37). The basic region (i.e., amino acids 47-57) is thought to be important for nuclear localization. Ruben, S. et al., J. Virol. 63: 1-8 (1989); Hauber, J. et al., J. Virol. 63 1181-1187 (1989); Rudolph et al. (2003) 278(13):11411. The cysteine-rich region mediates the formation of metal-linked dimers in vitro (Frankel, A. D. et al., Science 240: 70-73 (1988); Frankel, A. D. et al., Proc. Natl. Acad. Sci USA 85: 6297-6300 (1988)) and is essential for its activity as a transactivator (Garcia, J. A. et al., EMBO J. 7:3143 (1988); Sadaie, M. R. et al., J. Virol. 63: 1 (1989)). As in other regulatory proteins, the N-terminal region may be involved in protection against intracellular proteases (Bachmair, A. et al., Cell 56: 1019-1032 (1989).

In one embodiment of the invention, tat protein is used to deliver cdx. In one embodiment of the invention, tat protein is used to deliver hox. In one embodiment of the invention, the basic peptide comprises amino acids 47-57 of the HIV-1 TAT peptide. In another embodiment, the basic peptide comprises amino acids 48-60 of the HIV-1 TAT peptide. In still another embodiment, the basic peptide comprises amino acids 49-57 of the HIV-1 TAT peptide. In yet another embodiment, the basic peptide comprises amino acids 49-57, 48-60, or 47-57 of the HIV-1 TAT peptide, does not comprise amino acids 22-36 of the HIV-1 TAT peptide, and does not comprise amino acids 73-86 of the HIV-1 TAT peptide.

The hematopoietic stem cells generated by the methods of the invention can be used for a variety of applications, including transplantation, sometimes referred to as cell-based therapies or cell replacement therapies, such as bone marrow transplants, gene therapies, tissue engineering, and in vitro organogenesis. The cell populations may be used for screening various additives for their effect on growth and the mature differentiation of the cells. In this manner, compounds which are complementary, agonistic, antagonistic or inactive may be screened, determining the effect of the compound in relationship with one or more of the different cytokines.

The populations may be employed as grafts for transplantation. For example, hematopoietic cells are used to treat malignancies, bone marrow failure states and congenital metabolic, immunologic and hematologic disorders. Marrow samples may be taken from patients with cancer, and enriched populations of hematopoietic stem cells isolated by means of density centrifugation, counterflow centrifugal elutriation, monoclonal antibody labeling and fluorescence activated cell sorting. The stem cells in this cell population are then expanded in vitro and can serve as a graft for autologous marrow transplantation. The graft will be infused after the patient has received curative chemo-radiotherapy.

Hematopoietic progenitor cell expansion for bone marrow transplantation is a potential application of human long-term bone marrow cultures. Human autologous and allogeneic bone marrow transplantation are currently used as therapies for diseases such as leukemia, lymphoma, and other life-threatening diseases. For these procedures, however, a large amount of donor bone marrow must be removed to ensure that there are enough cells for engraftment. The methods of the present invention circumvent this problem. Methods of transplantation are known to those skilled in the art.

Hematopoeitic stem cells generated by the methods of the invention are particularly suited for reconstituting hematopoietic cells in a subject or for providing cell populations enriched in desired hematopoietic cell types. This method involves administering by standard means, such as intravenous infusion or mucosal injection, the expanded cultured cells to a patient. Intravenous administration also affords ease, convenience and comfort at higher levels than other modes of administration. In certain applications, systemic administration by intravenous infusion is more effective overall. In a preferred embodiment, the stem cells are administered to an individual by infusion into the superior mesenteric artery or celiac artery. The cells may also be delivered locally by irrigation down the recipient's airway or by direct injection into the mucosa of the intestine.

After isolating the cells, the cells can cultured for a period of time sufficient to allow them to expand to desired numbers, without any loss of desired functional characteristics. For example cells can be cultured from 1 day to over a year. Preferably the cells are cultured for 3-30 days, more preferably 4-14 days, most preferably at least 7 days.

Between 105 and 1013 cells per 100 kg person are administered per infusion. Preferably, between about 1-5×108 and 1-5×1012 cells are infused intravenously per 100 kg person. More preferably, between about 1×109 and 5×1011 cells are infused intravenously per 100 kg person. For example, dosages such as 4×109 cells per 100 kg person and 2×1011 cells can be infused per 100 kg person.

In some embodiments, a single administration of cells is provided. In other embodiments, multiple administrations are used. Multiple administrations can be provided over periodic time periods such as an initial treatment regime of 3-7 consecutive days, and then repeated at other times.

With respect to cells as administered to a patient in vivo, an effective amount may range from as few as several hundred or fewer to as many as several million or more. In specific embodiments, an effective amount may range from 103-1011. It will be appreciated that the number of cells to be administered will vary depending on the specifics of the disorder to be treated, including but not limited to size or total volume/surface area to be treated, as well as proximity of the site of administration to the location of the region to be treated, among other factors familiar to the medicinal biologist.

The terms effective period (or time) and effective conditions refer to a period of time or other controllable conditions (e.g., temperature, humidity for in vitro methods), necessary or preferred for an agent or pharmaceutical composition to achieve its intended result.

The term pharmaceutically acceptable carrier (or medium), which may be used interchangeably with the term biologically compatible carrier or medium, refers to reagents, cells, compounds, materials, compositions, and/or dosage forms which are, within the scope of sound medical judgment, suitable for use in contact with the tissues of human beings and animals without excessive toxicity, irritation, allergic response, or other complication commensurate with a reasonable benefit/risk ratio. As described in greater detail herein, pharmaceutically acceptable carriers suitable for use in the present invention include liquids, semi-solid (e.g., gels) and solid materials (e.g., cell scaffolds). As used herein, the term biodegradable describes the ability of a material to be broken down (e.g., degraded, eroded, dissolved) in vivo. The term includes degradation in vivo with or without elimination (e.g., by resorption) from the body. The semi-solid and solid materials may be designed to resist degradation within the body (non-biodegradable) or they may be designed to degrade within the body (biodegradable, bioerodable). A biodegradable material may further be bioresorbable or bioabsorbable, i.e., it may be dissolved and absorbed into bodily fluids (water-soluble implants are one example), or degraded and ultimately eliminated from the body, either by conversion into other materials or by breakdown and elimination through natural pathways.

Several terms are used herein with respect to transplantation therapies, also known as cell-based therapies or cell replacement therapy. The terms autologous transfer, autologous transplantation, autograft and the like refer to treatments wherein the cell donor is also the recipient of the cell replacement therapy. The terms allogeneic transfer, allogeneic transplantation, allograft and the like refer to treatments wherein the cell donor is of the same species as the recipient of the cell replacement therapy, but is not the same individual. A cell transfer in which the donor's cells have been histocompatibly matched with a recipient is sometimes referred to as a syngeneic transfer. The terms xenogeneic transfer, xenogeneic transplantation, xenograft and the like refer to treatments wherein the cell donor is of a different species than the recipient of the cell replacement therapy.

The expanded hematopoietic cells can be used for reconstituting the full range of hematopoietic cells in an immunocompromised host following therapies such as, but not limited to, radiation treatment and chemotherapy. Such therapies destroy hematopoietic cells either intentionally or as a side-effect of bone marrow transplantation or the treatment of lymphomas, leukemias and other neoplastic conditions, e.g., breast cancer.

Expanded hematopoietic cells are also useful as a source of cells for specific hematopoietic lineages. The maturation, proliferation and differentiation of expanded hematopoietic cells into one or more selected lineages may be effected through culturing the cells with appropriate factors including, but not limited to, erythropoietin (EPO), colony stimulating factors, e.g., GM-CSF, G-CSF, or M-CSF, SCF, interleukins, e.g., IL-1, -2, -3, -4, -5, -6, -7, -8, -13, etc., or with stromal cells or other cells which secrete factors responsible for stem cell regeneration, commitment, and differentiation.

Expanded hematopoeitic cells of the invention are useful for identifying culture conditions or biological modifiers such as growth factors which promote or inhibit such biological responses of stem cells as self-regeneration, proliferation, commitment, differentiation, and maturation. In this way-one may also identify, for example, receptors for these biological modifiers, agents which interfere with the interaction of a biological modifier and its receptor, and polypeptides, antisense polynucleotides, small molecules, or environmental stimuli affecting gene transcription or translation.

For example, the present invention makes it possible to prepare relatively large numbers of hematopoietic stem cells for use in assays for the differentiation of stem cells into various hematopoietic lineages. These assays may be readily adapted in order to identify substances such as growth factors which, for example, promote or inhibit stem cell self-regeneration, commitment, or differentiation.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

### EXAMPLES

### Example 1

### Embryonic Stem Cell Derived Hematopoietic Stem Cells

### Materials and Methods

### Cell Culture:

ESCs were maintained on mitomycin C-treated mouse embryonic fibroblasts (MEFs, Specialty Media) in DME/15% FBS, 0.1 mM nonessential amino acids (GIBCO), 2 mM glutamine, 500 u/ml penicillin/streptomycin (GIBCO), 0.1 mM β-mercaptoethanol, and 1000 U/ml LIF (Peprotech). ESCs were differentiated in vitro and infected by retrovirus according to published protocols (Kyba, 2002). Briefly, ESC cultures were depleted of MEFs by differential adhesion after incubation in tissue culture flasks for 35 minutes (during which time the MEFs adhere) and were then plated as 10 ul hanging drops in EB differentiation media (IMDM/15% fetal bovine serum [StemCell Technologies], 200 ug/ml iron-saturated transferrin [Sigma], 4.5 mM monothioglycerol [Sigma], 50 ug/ml ascorbic acid [Sigma], and 2 mM glutamine) for two days. EBs were harvested from hanging drops at day 2 and transferred into 10 ml EB differentiation media in slowly rotating 10 cm petri dishes for another 4 days. Doxycycline was added from day 3 to day 4 at 0.1 ug/ml and from day 4 to 6 at 0.5 ug/ml as the final concentration to induce cdx4 expression. Cells were harvested at day 6 after collagenase treatment. A total of 105 EB cells were plated onto semiconfluent OP9 cells in 6-well dishes and were infected with retroviral supernatants, which were produced in 293 cells by FUGENE (Roche) cotransfection of viral plasmid MSCV-HoxB4-ires-GFP and packaging-defective helper plasmid, pCL-Eco (Kyba, 2002). Infected EB cells were grown in 3 ml of IMDM/10% inactivated fetal bovine serum (IFS) with cytokines (100 ng/ml SCF, 40 ng/ml VEGF, 40 ng/ml TPO, 100 ng/ml Flt-3 ligand). When confluent, the cultures were passed by pooling suspension and semiadherent cells (obtained by trypsinization) and replated onto fresh OP9.

### Generation of Tetracycline-Inducible Cdx4 ES Cell Line

The murine Cdx4 cDNA (a kind gift from Dr. Alan Davidson) was inserted into the EcoRI/XbaI treated site of plox (Kyba, 2002). The parental ES cell line Ainv15 (Kyba, 2002) was targeted with plox-cdx4 by coelectroporation of 20 ug each of plox-cdx4 and the Cre recombinase expression plasmid, p00231 (O'Gorman, 1997), followed by selection in ES medium with 350 ug/mL G418 (GIBCO) and isolation of positive clones to generate the inducible cell line, icdx4. The induction of cdx4 expression upon doxycycline treatment was confirmed by RT-PCR on total RNA collected from positive clones.

### Blast Colony Formation and Replating Assay

Blast cell colonies were generated as previously described (Kennedy, 1997). Briefly, in this study, 3×104Cells from EB after 3.2 days of differentiation were collected by collagenase treatment and plated into 1.5 ml of methycellulose medium (13120, StemCell Technologies) with 10% IFS, 25 ug/ml ascorbic acid, 200 μg/ml iron-saturated transferring; 5 ng/ml VEGF (Peprotech), and 4.5×10−4 M monothioglycerol (MTG). Colonies were scored 4 days after plating. For generation of secondary hematopoietic colonies, individual blast colony was picked and plated into methycellulose medium (M3434, StemCell Technologies). Hematopoietic colonies were then scored between day 6 to 10 post plating. For endothelial replating, individual blast colonies were transferred to matrigel-coated Lab-Tek chamber slides (Nalge Nunc International) containing IMDM with 10% fetal calf serum (Hyclone), 10% horse serum (Gibco), VEGF (5 ng/ml), IGF-1 (Peprotech, 10 ng/ml), bFGF (Peprotech, 10 ng/ml), endothelial cell growth supplement. (ECGS, 100 mg/ml; Collaborative Research), L-glutamine (2 mM), and 4.5×10−4 M MTG. Following 2-3 weeks in culture, cells were either harvested for preparation for total RNA or for fluorescence analysis as previously described (Kennedy, 1997; Choi, 1998).

### RT-PCR Analysis and Quantitative Real-Time PCR

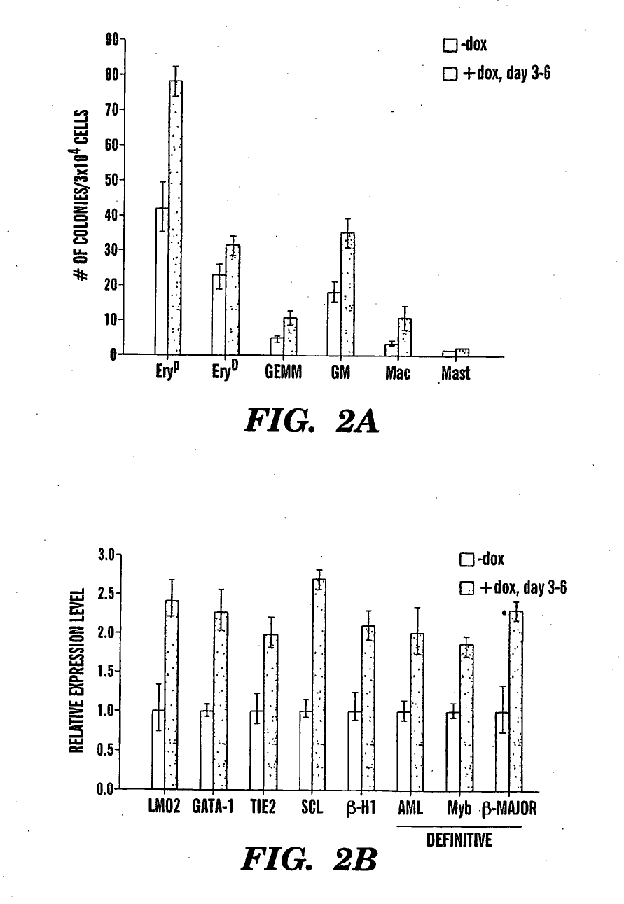

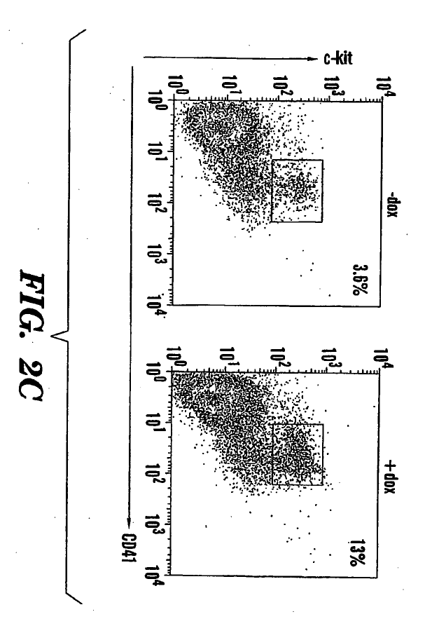

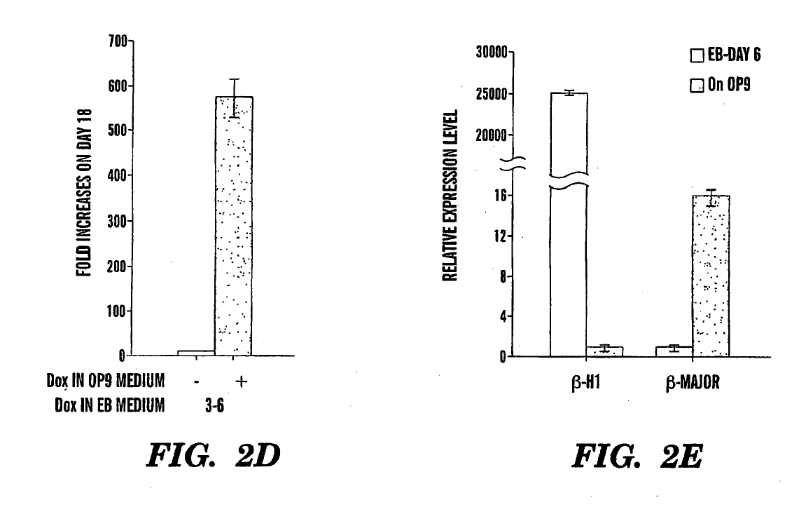

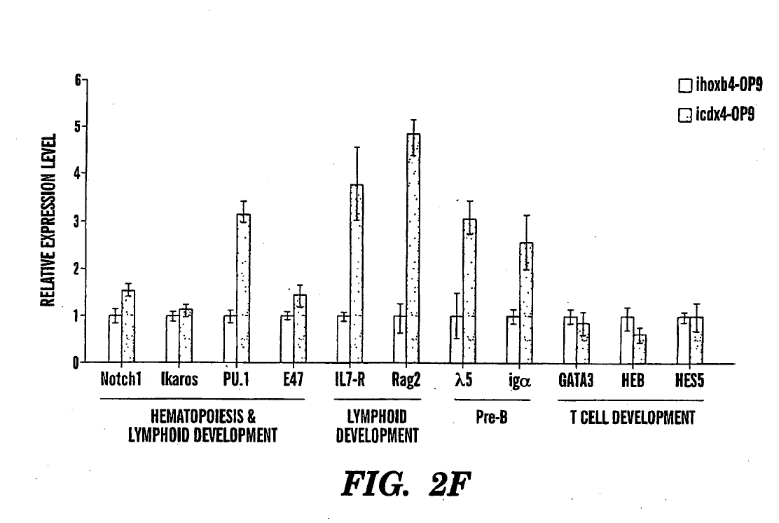

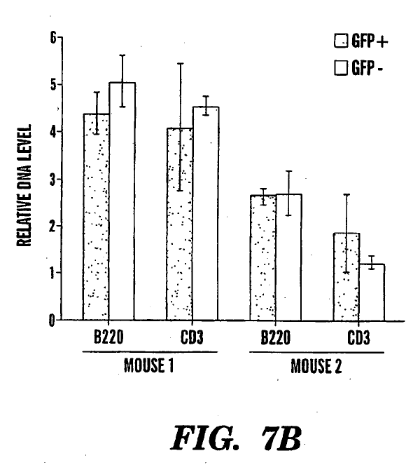

Cells were harvested in RNA Stat-60 and total RNA was isolated according manufacture's instruction (Tel-Test Inc.). All RNA samples were treated with DNaseI and then purified by RNeasy MinElute kit (Qiagen). cDNA were prepared according the manufacture's instruction (Invitrogene). Real-time PCR was performed in triplicates with a SYBR green PCR kit (Applied Biosystems) according to manufacturer's protocol on an ABI Prism 7700 Sequence Detector. For experiments in FIG. 7A and FIG. 7B, GFP DNA levels are quantified into arbitrary units using the comparative CT method (relative to the TDAG51 gene as an internal normalization control) (Livak, 2001). For FIGS. 2B & 2F, test gene expression was normalized to β-actin and relative expression levels were derived with the comparative CT method.

### Cell Transplantation

Six week to three month-old Rag2−/−/γc−/− female mice were given two doses of 400 cGy γ-irradiation, separated by 4 hours; and were injected via lateral tail vein with 2×106 cells in 400 ul IMDM/2% IFS. Transplanted mice were maintained under sterile conditions.

### FACS Analysis

Prior to FACS analysis, peripheral blood leukocytes, splenocytes and bone marrow were treated with red cell lysis buffer (Sigma). All antibodies used in FACS analysis or sorting were purchased from Pharmingen, BD Biosciences. Propidium iodide was added to exclude dead cells. Gr1+, B220+, or CD3+ cells were isolated either by FACS sorting or by positive selection with magnetic streptavidin-conjugated Dynabeads M280 according to the manufacturer's protocol (Dynal Biotech). The purity of sorted cells was checked by post-sorting FACS analysis.

Genomic DNA isolation and Southern Hybridization

GFP and HoxB4 probes were obtained separately by purification of an NcoI/ClaI digested fragment from MSCV-ires-GFP and an EcoRI/XhoI fragment from MSCV-HoxB4-ires-GFP with MinElu gel purification kit (Qiagen). Probes were then labeled with α³²P-dCTP with a random primer labeling kit (Stratagene). Genomic DNA was isolated with a genomic DNA purification kit from Gentra Systems according to the manufacturer's protocol. Restriction digestion and electrophoresis were carried out according to standard procedures. DNA separated by gel electrophoresis was transferred onto Hybond/N+ nylon membrane (Amersham), and hybridized with ³²P-labelled probe in Miraclehyb solution (Stratagene) at 65° C. overnight, washed twice with 0.5×SSC/0.1% SDS for 10 minutes at room temperature and twice with 0.1×SSC/0.1% SDS for 15 minutes at 65° C. in a shaking water bath, and then rinsed with 2×SSC. The target DNA was finally visualized by phosphorimaging, and band intensity was measured by ImageQuant and Adobe Photoshop software. The first GFP probe was stripped from the membrane in boiling 0.1×SSC/0.1% SDS before hybridization with the second HoxB4 probe.

### Results

### Establishing a Tetracycline Inducible Cdx4 Cell Line

In order to achieve a consistent, titratable and homogenous induction of Cdx4 and to enable reversible Cdx4 expression in the in vitro ESC differentiation system as well as in engrafted animals, mouse Cdx4 cDNA was cloned into an inducible transgene system. In this system, Cdx4 was integrated near the HPRT gene on the X-chromosome under the control of a tetracycline responsive promoter element. RT-PCR was performed with Cdx4 specific primers on total RNA isolated from positive ESC colonies and showed that Cdx4 was induced significantly upon the treatment of doxycycline (dox) for 24 hours.

### Cdx4 Enhances Hemangioblast Formation

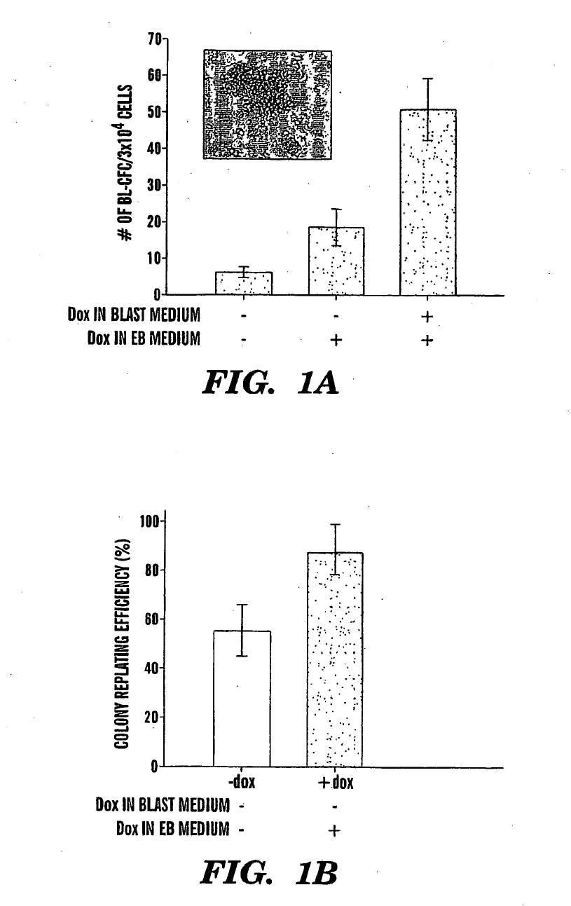

Our previous study showed that the expression peak of Cdx4 was restrictively from day 3 to day 4 during embryoid body (EB) development in vitro (Davidson, 2003). This time period corresponds to the emergency of hematopoietic mesoderm such as hemangioblast during EB differentiation. Hemangioblast is a common precursor of hematopoietic and endothelial lineages from mesoderm arisen from EB (Kennedy, 1997; Choi, 1998). Therefore, we examined whether Cdx4 could promote hemangioblast formation. As shown in FIG. 1A, induction of Cdx4 with doxycycline from day 2 to day 3.2 during EB development and/or in the blast media enhanced hemangioblast forming-frequency. Individual blast colonies were picked and replated into methycellulose M3434 (to detect blood progenitor formation) and endothelial growth media. Approximately 60% of blast colonies formed 2^{nd} blood progenitor colonies in non-induced cells and induction of Cdx4 increased the replating efficiency of blast cells into hematopoietic colonies (FIG. 1B). In the endothelial replating experiment, fibroblast cell line 3T3 and endothelial cell line D4T were used as the negative control and the positive control respectively. As shown by immunofluorescence, only cells from D4T and replated blast cells demonstrated the characteristics of endothelial cells, which expressed CD31 and uptook acetylated low-density-lipoprotein (Dil-LDL). In addition, cells harvested from blast colony also expressed endothelial markers such as flk1 and Tie2. Therefore, the replating experiments confirmed the blast colonies we observed in the colony forming assay were true hemangioblast colonies. Taken together, we concluded from these data that Cdx4 could promote hematopoietic mesoderm specification from differentiated ES cells.

### Cdx4 Promotes Both Primitive and Definitive Hematopoiesis In Vitro

By conditionally inducing Cdx4 expression from day 3 to 6 of EB differentiation, we observed a marked enhancement of primitive erythroid and multipotential hematopoietic colonies (FIG. 2A). CD41 and c-kit are markers for early hematopoietic progenitors in embryos and EBs. As shown in FIG. 2C, compared with non-induced cells, percentage of CD41⁺/c-kit⁺ cells was increased in day 6 EB population exposed to doxycycline, suggesting that Cdx4 promoted hematopoietic colony formation by enhancing the proliferation of clonogenetic hematopoietic progenitors. Consistent with this finding, by using real-time RT-PCR, we demonstrated that the expression level of genes involved in hematopoiesis was elevated 2- to 3-fold in day 6 EB with Cdx4 activation (FIG. 2B). The difference in expression level of these genes was indiscernible within CD4′-sorted day 6 EB cells from non-induced and doxcycline-induced population (data not shown). Therefore, these genes may not be true target genes of Cdx4; instead, the enhanced gene expression might correlate to an increased percentage of hematopoietic cells in whole EB by Cdx4 activation. Of the genes we assayed, β-H1, Tie2, LMO2, SC1, and GATA1 were involved in both early hematopoietic development and certain definitive lineage differentiation, while β-major, c-myb, and AML, were markers for definitive hematopoiesis. Elevated expression of these genes suggests that Cdx4 activation promoted both primitive and definitive hematopoietic progenitor formation from differentiated ESCs.

OP9 is a stromal cell line derived from M-CSF deficient mice and supports the growth of hematopoietic progenitors (Nakano, 1994). Under our culture condition, non-induced day 6 EB cells failed to expand on OP9 cells. In contrast, ectopic expression of Cdx4 induced by doxycycline enabled EB-derived hematopoietic blasts to expand and undergo multi-lineage differentiation on OP9 (FIG. 2D & Table 1). Moreover compared to day 6 EB, expression of β-H1 embryonic globin was significantly reduced, while the expression of β-major, the adult-type globin, was elevated in OP9 co-cultured cells, indicating Cdx4 enabled definitive erythroid progenitors growing on OP9 (FIG. 2E).

TABLE 1
Surface antigene expression of ESC-derived
cells growing on OP9 for 23 days

| Lineage | Surface Marker | icdx4/+dox | ihoxB4/+dox |
|---|---|---|---|
| Myeloid | Gr-1 | | |
| | Mac-1 | | |
| Erythroid | Ter119 | 0.62 | 0.36 |
| Lymphoid | CD4 | 0.1 | 0 |
| | CD8 | 0 | 0 |
| | B220 | 7.21 | 0.70 |
| Progenitor/Meg | CD41 | 85.94 | 89.94 |
| HSC | Sca1 | 43.63 | 16.16 |
| | c-kit | 96.7 | 75.7 |
| | c-kit/Sca1 | 42.1 | 12.1 |
| HSC/Endothelial | CD31 | 98.36 | 85.06 |
| | CD34 | 92.22 | 10.25 |
| | flk-1 | 0.31 | 0.26 |
| | VE-Cadherin | 71.73 | 51.23 |
| | Endoglin | 68.83 | 53.43 |
| Pan-hematopoietic | CD45 | 85.3 | 50.4 |

Because HoxB4 expression also led to an expansion of EB-derived hematopoietic cells on OP9 as demonstrated in our previous study, we compared the surface antigen and gene expression profile of OP9 co-cultured cells expanded by HoxB4 to those derived from inducible Cdx4 cell line. Of the surface antigens positive for lineages we assayed, cells overexpressing Cdx4 have higher percentage of B220+ cells than HoxB4-expanded cells. This observation is consistent with the gene expression profile, in which the expression of certain genes involved in lymphoid development, especially in B cell development were increased in Cdx4 induced OP9 co-cultured cells (FIG. 2F). Majority of Cdx4-expanded cells were CD31+/CD34+/c-kit+/CD41+. The percentage of CD45+ cells increased from 50% at day 6 to 90% after 20 days of culture. Eventually most of cells (>85%) became CD31+/CD41+/CD34+/c-kit+/CD45+, and half of them was also Sca1+. Cells with overexpression of HoxB4 have a similar percentage of CD41+ cell as those expanded by Cdx4. The percentage of CD31+ was lower at the beginning (day 6 after plating), but soon reached to 85%. After 23 days in culture, majority of HoxB4-expanded cells was CD31+/CD41+, among them, only half of the cells were CD45+., and most of them were CD34-. CD45 is used as adult pan-hematopoietic marker. The expression of CD45 is developmentally regulated and appears later than CD41 in embryo and EB. In day 9.5 YS and day 6 EBs, the CD45+ cells are first detected in a subpopulation of CD41+. Hematopoietic progenitor colony forming potential existed in both CD45+/CD41+ and CD45+/CD41+ cells. But soon in fetal liver stage, colony forming potential shifts to CD45+, with downregulation of CD41 expression, suggesting that. CD45+/CD41+ double positivity may be an intermediate stage of embryonic hematopoietic population to acquire a definitive phenotype. Therefore, higher percentage of CD45+ cells in Cdx4-expaned population suggested Cdx4 could drive the switching of primitive to definitive hematopoiesis more efficiently than HoxB4. CD34 is developmentally and functionally regulated, and its expression is influenced by the activation-state of stem cells. Higher expression of CD34+ in Cdx4 expanded cells suggested these cells were at actively cycling state undergoing proliferation and differentiation.

### Cdx4 Improves Engraftment of ES-Derived Hematopoietic Progenitors

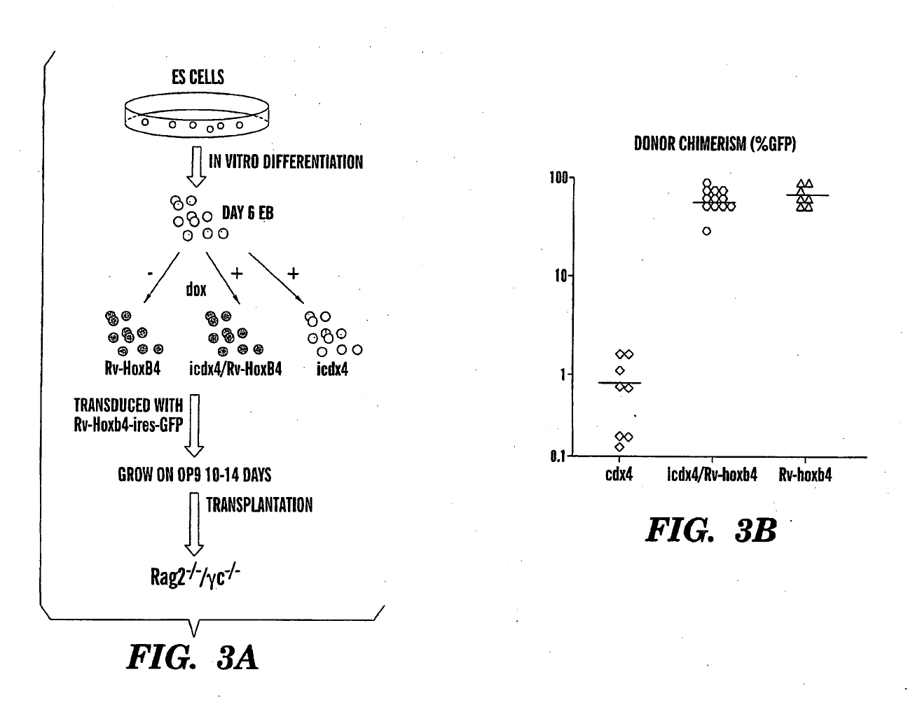

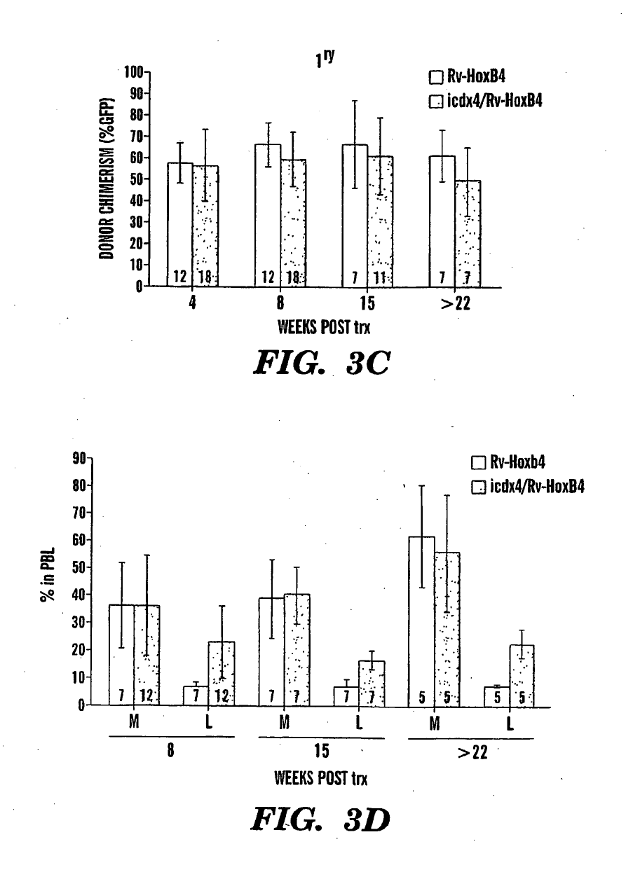

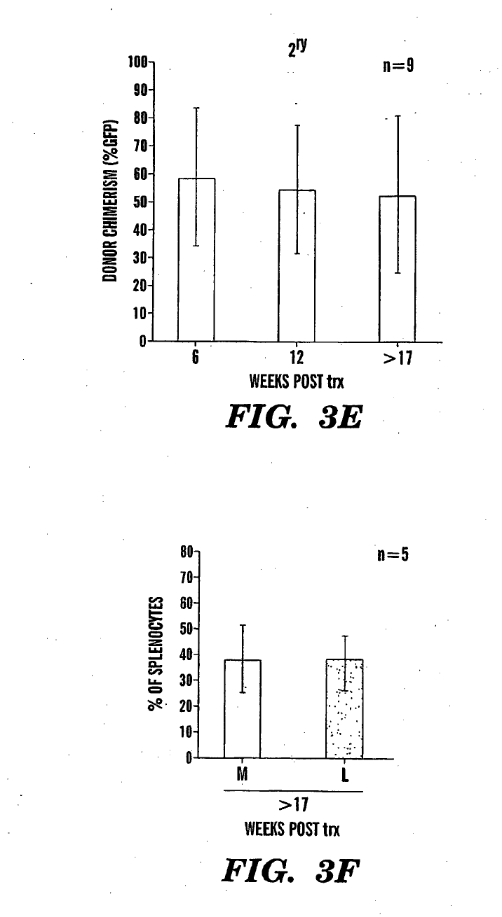

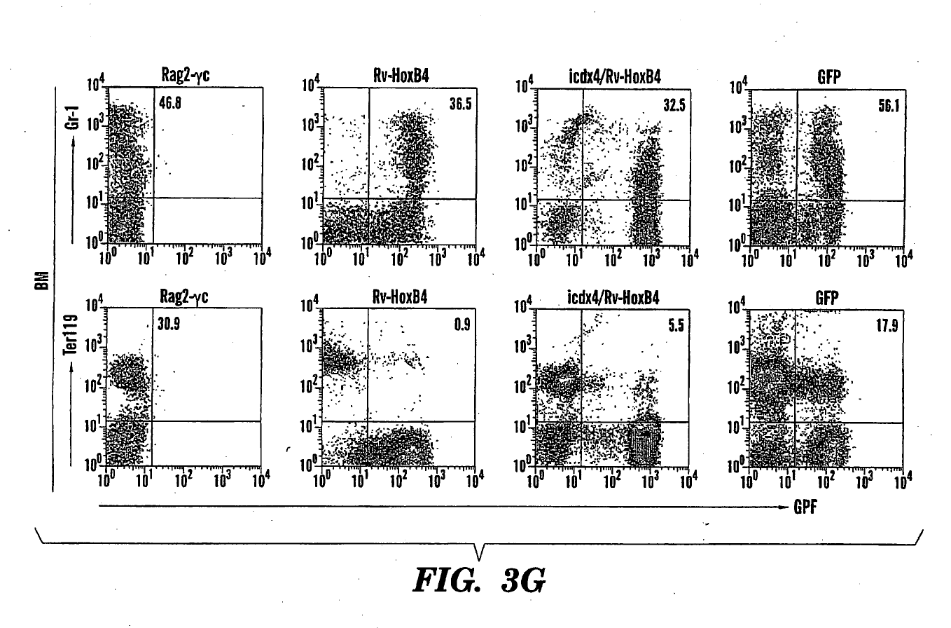

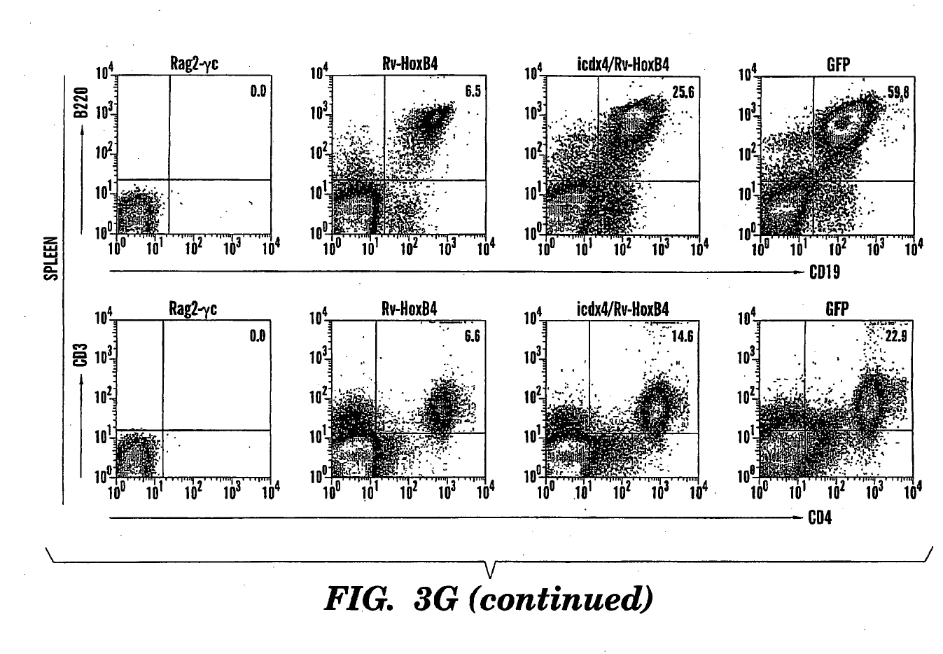

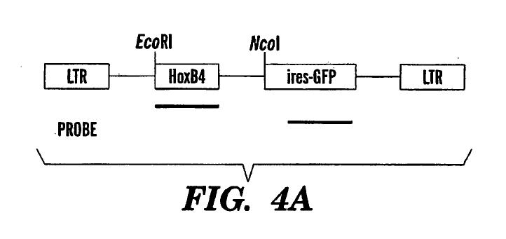

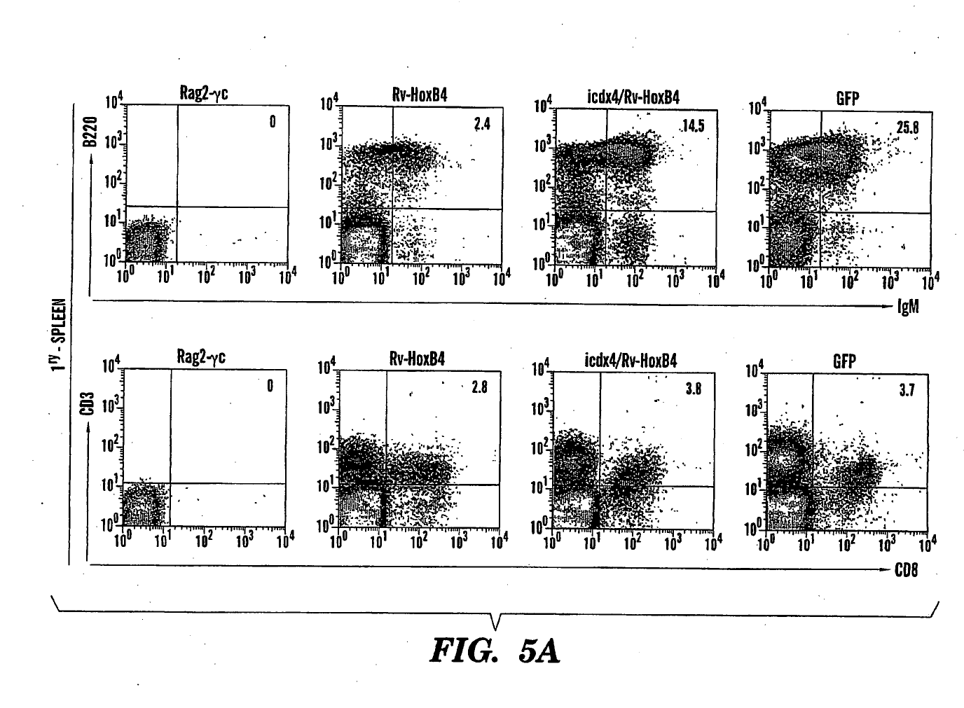

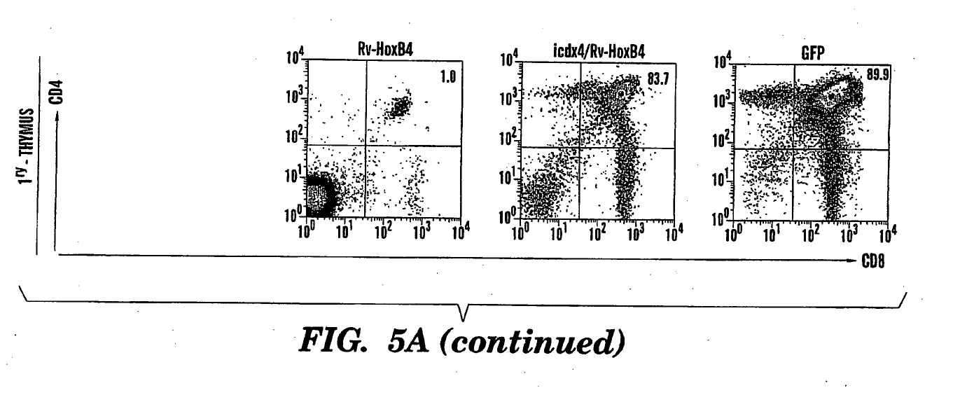

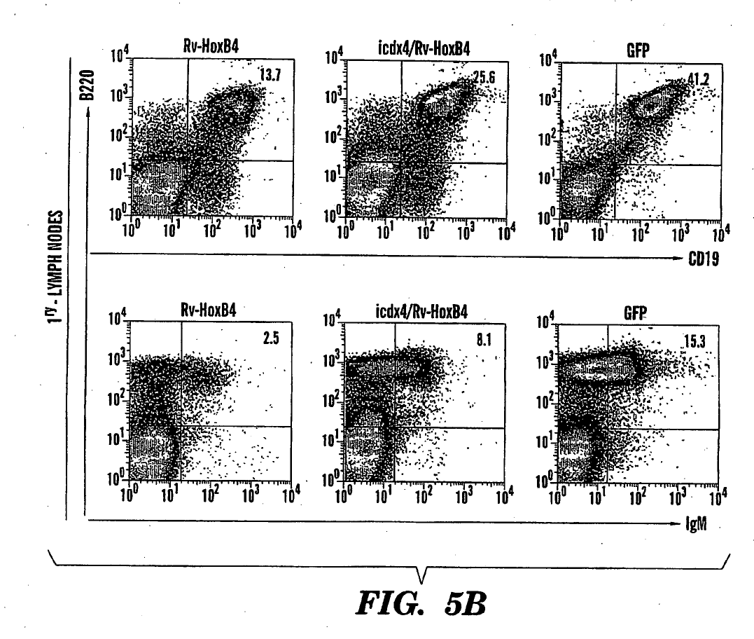

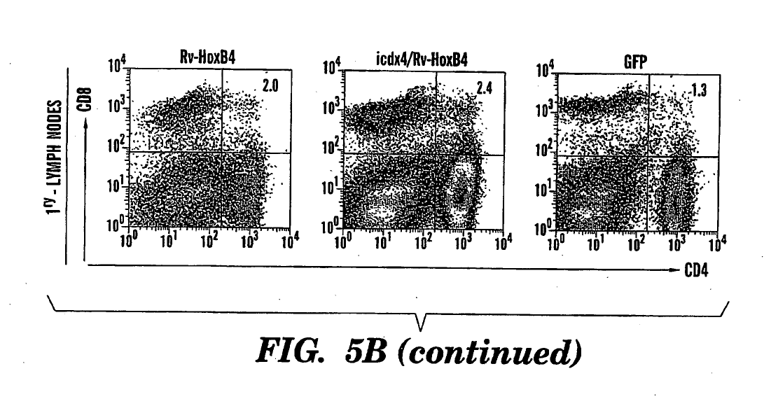

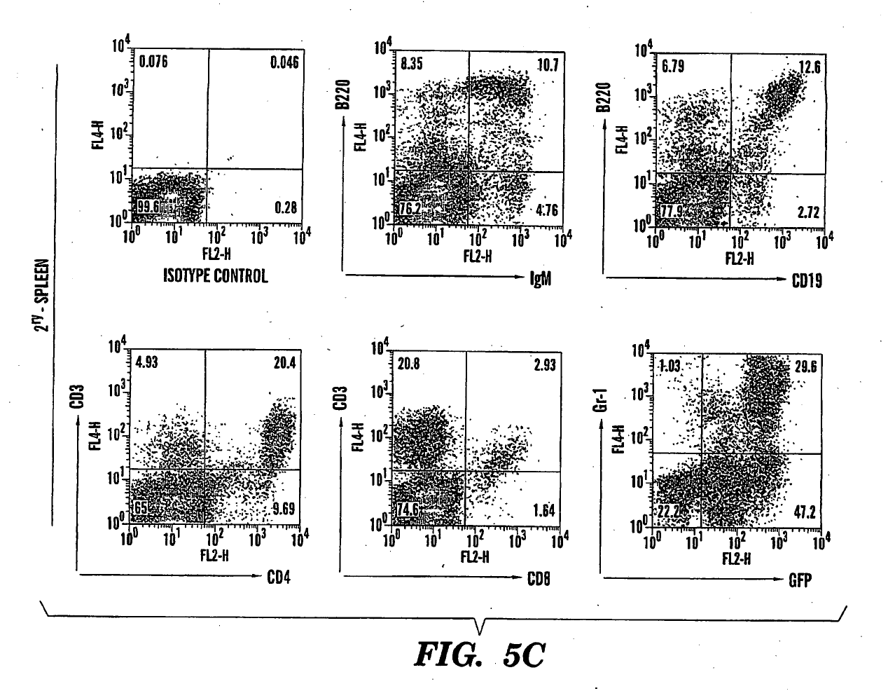

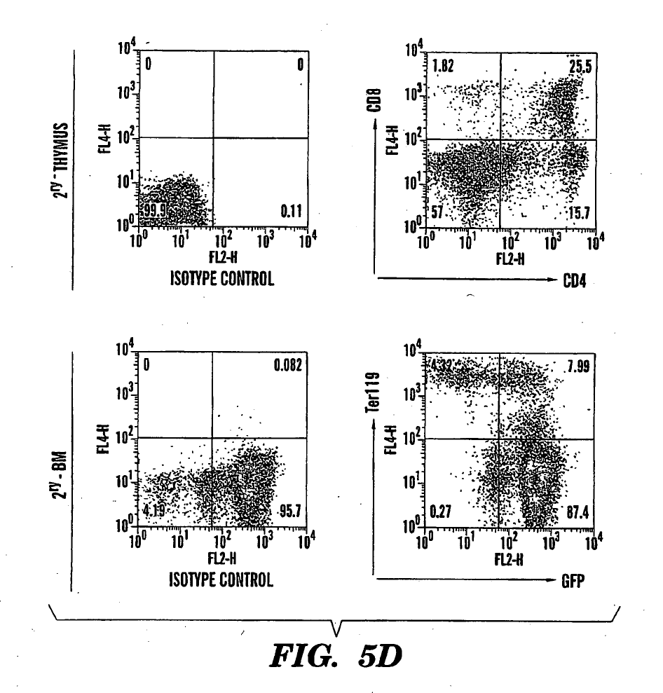

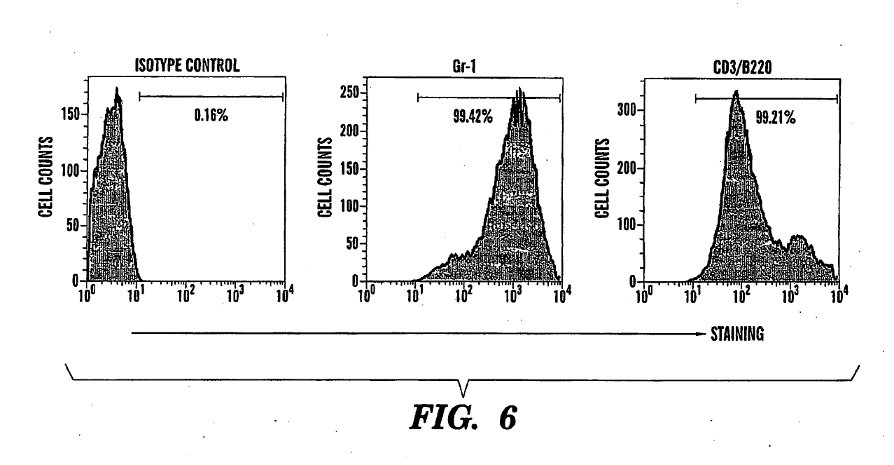

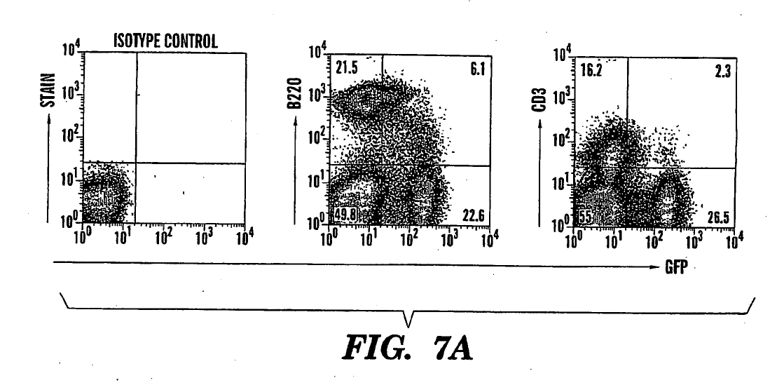

Results described above demonstrated that ectopic expression of Cdx4 increased CD41+/c-kit+ cells from EB and enhanced the expression of genes involved in definitive hematopoiesis and lymphoid development. In addition, multilineage differentiation and 3-globin switching to adult-type of hematopoietic blasts on OP9 suggested the existence of definitive hematopoietic progenitors in Cdx4-induced cell population. Therefore, we next explored whether Cdx4 could improve engraftment of ES-derived hematopoietic progenitors into lethally irradiated mice. As shown in FIG. 3B, ESC-derived hematopoietic progenitors with ectopic expression of Cdx4 engrafted mice only with low level of radioprotection (8 out of 30 survived after 8 weeks post transplantation) and donor chimerism (average<1%), suggesting that the transplanted population only contained small number of definitive HSCs or only progenitors which had only limited self-renewal potential. It is possible that Cdx4 alone does not have sufficient self-renewal ability to maintain ESC-derived HSCs to grow on OP9 stromal cells for a long time. Thus we next examined if combination of hematopoietic specification ability of Cdx4 with self-renewal potential of HoxB4 could improve better engraftment from differentiated ESCs. EBs were formed from a inducible Cdx4 cell line. The expression of Cdx4 was induced by doxycycline during day 3 to 6 of EB development (timed to coincide with the specification of blood lineages from ESCs), while a separate population of EBs was left uninduced. Day 6 EB cells from both groups were transduced with a retroviral vector expressing HoxB4 linked via IRES (Internal Ribosomal Entry Site) (Mountford, 1994) to Green Fluorescent Protein (GFP), and grown on OP9 stromal cells for 10-14 days, as described in FIG. 3A (Kyba, 2002; Nakano, 1994). Cultured cells were then injected intravenously into lymphocyte-deficient Rag2/γc double knockout mice (Colucci, 1999) that had been lethally irradiated (800 cGy). Hematopoietic populations modified by either HoxB4 alone or by Cdx4 plus HoxB4 successfully reconstituted blood formation in otherwise lethally-irradiated mice. In data from three independent transplantation experiments, survival due to the radio-protective effect of transplanted cells was close to 100% at 8 weeks (12/13 for HoxB4; 18/18 for Cdx4/HoxB4). Flow cytometric monitoring of GFP+ cells in the peripheral blood of transplanted animals showed high-level donor chimerism that was stable over 6 months (FIG. 3A). Moreover, myeloid, lymphoid, and erythroid lineages were reconstituted in the peripheral blood, spleen, lymph nodes, bone marrow, and thymus of engrafted mice (FIGS. 3C, 3D, 3G, and 5A). Interestingly, when compared with mice transplanted with cells treated with HoxB4 alone, mice engrafted with Cdx4/HoxB4 treated cells consistently showed a higher degree of lymphoid reconstitution (FIGS. 3D & 3G). Thus, this experiment is consistent the in vitro experiment results described above and suggests induction of Cdx4 during EB differentiation promoted transplantable HSCs formation. In addition, these data established conditions for robust and reproducible hematopoietic engraftment of lethally irradiated mice with the hematopoietic progeny of ESCs differentiated in vitro.

Bone marrow from primary animals engrafted with Cdx4/HoxB4-expressing cells successfully reconstituted multiple lineages of hematopoietic cells when transplanted into lethally irradiated secondary mice (FIGS. 3E, 3F and 3B). Moreover, the thymus from both primary and secondary engrafted animals was reconstituted with CD4+/CD8+ cells for more than four months post-transplantation (FIGS. 5A and 5B), indicating stable and long-term engraftment of the lymphoid lineage. Taken together, the existence of CD4+/CD8+ double-positive cells in the thymus of both primary and secondary engrafted mice, and the detection of the expected blood lineages in the peripheral blood, spleen, lymph nodes, bone marrow and thymus suggested stable hematopoietic reconstitution with self-renewing, multipotential HSCs.

### Clonal Analysis of ESC-HSC Engrafted Population

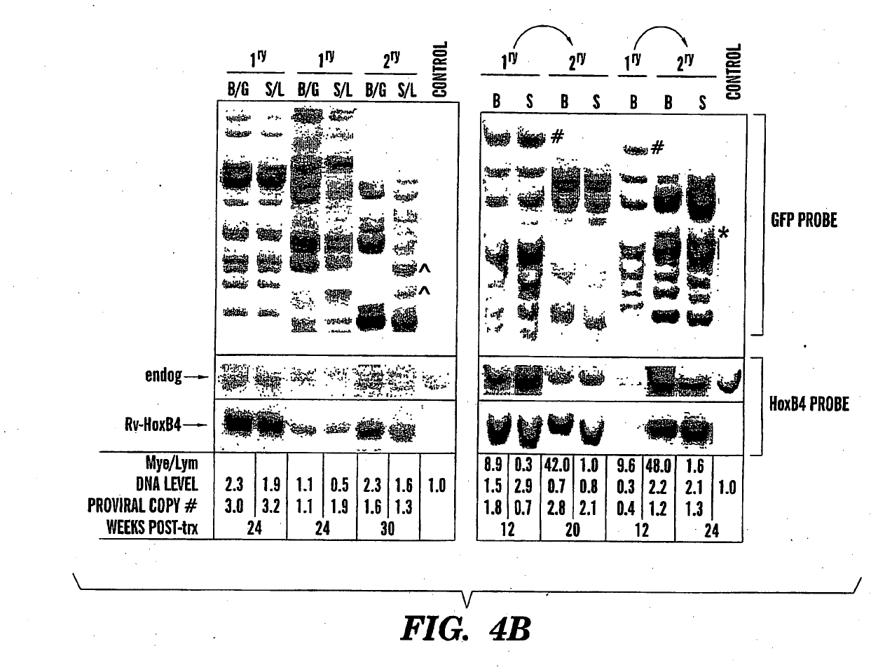

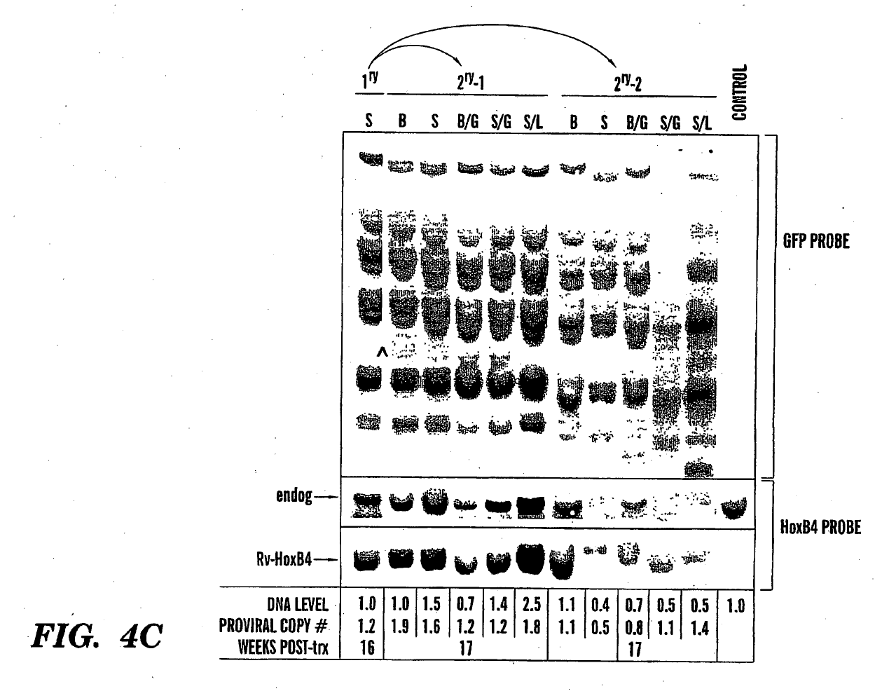

Clonal analysis of marked donor cells has been used as the gold standard for documenting the BM-HSC (Keller, 1985; Lemischka, 1986) and the introduction of HoxB4 via retrovirus into the ESC-derived hematopoietic populations allowed us to use the proviral integration site as a unique genetic marker (FIG. 4A). Genomic DNA was isolated from either spleen or bone marrow cells of primary and secondary mice. In some cases, genomic DNA was extracted from populations of Gr-1⁺ myeloid cells and B220⁺ and CD3⁺ lymphoid cells that were purified by antibody-conjugated magnetic beads or low cytometric sorting to >99% homogeneity. Isolated DNA was digested with EcoRI and NcoI, resolved by agarose gel electrophoresis, and analyzed by Southern hybridization with probes that reflected either the unique proviral integration site (GFP) or the fragment of the HoxB4 cDNA common to all proviruses (as well as endogenous HoxB4, which served as an internal DNA loading control). In essentially all samples tested, we detected multiple co-migrating fragments (bands), representing shared proviral integration sites in cells from spleen and bone marrow, and from fractionated myeloid and lymphoid cell populations from primary and secondary mice (FIGS. 4B and 4C). Importantly, several co-migrating fragments were seen in paired primary and secondary mice after long-term engraftment (>17 weeks), indicating that multiple clones carried extensive self-renewal capacity (FIGS. 2B and 2C). Moreover, by comparing the hybridization intensity of the endogenous and proviral HoxB4 fragments, we calculated that most tissues harbored between 1-3 proviral copies/cell, and showed engraftment with 7-15 prominent clones (FIGS. 4B and 4C). Although most tissues harbor co-migrating bands, not all clones are represented among all tissues in paired samples. Some clones were seen only in primary recipients (FIG. 4B, #), others were unique to secondary engrafted animals (FIG. 4B, *), and some were seen predominantly in one lineage (FIGS. 4B and 4C, ̂). Such clonal extinction, clonal succession, and lineage restriction is an expected feature of HSC dynamics (Jordan, 1990).

### Discussion

In the present study, we demonstrated an important role of Cdx4 in specifying hematopoietic fate from differentiated ES cells by utilizing a tetracycline-inducible Cdx4 ES cell line. Overexpression of Cdx4 enhanced hematopoietic mesoderm, the hemangioblast, and multipotential hematopoietic progenitor formation in vitro. Moreover, conditional overexpression of Cdx4 enabled definitive hematopoietic progenitors to expand on OP9, and improved lymphoid engraftment from ES-derived hematopoietic progenitors, suggesting Cdx4 may also enhance the definitive HSC fate during ES differentiation.

Our previous study showed that ESC derived progenitors with ectopic HoxB4 expression engrafted mice with low level of lymphoid reconstitution. Comparing to cells expanded by HoxB4 on OP9 stromal cells, Cdx4 induced cells contained higher percentage of lymphoid cells. It is likely that Cdx4 has more potent ability to drive definitive hematopoietic progenitor formation, or HoxB4 has an inhibitory effect along lymphoid differentiation. However, the higher lymphoid reconstitution in the mice transplanted with cells treated with Cdx4/HoxB4 than HoxB4 alone can not be explained simply by inhibitory effect from HoxB4 because HoxB4 was expressed at similar level in both groups (data not shown); instead, it is plausible that Cdx4 induction increased percentage of definitive HSCs in the transplanted population.

The classical role of caudle-related family members is to act as master regulators of Hox gene expression in anterior-posterior pattering. Although the physiological function of Cdx4 has not been clearly understood during embryonic hematopoiesis in mammals, the downstream targets of Cdx4, several hox genes (such as HoxA6, HoxA9, HoxA10, HoxB4, and HoxB8) are indicated in normal and leukemic hematopoiesis; and cluster C (such as C6, the expression was also enhanced by Cdx4 activation) Hox genes were involved in lymphoid development. Moreover, Cdx4 overexpression can rescue the progenitor formation in Mll deficient ESCs; and Mll also Hox gene regulator involved in definitive hematopoiesis. Therefore, it is likely that the Hox gene patterning established by Cdx4 activation favors the hematopoietic specification, especially definitive hematopoiesis. It will be interesting to explore the Hox gene code during embryonic hematopoietic specification in the future studies. Cdx1 and/or Cdx2 knockout mice have been made. There is no significant defect in hematopoiesis in those animals, except the yolk sac circulation is abnormal in Cdx2 deficient embryos. This raises the possibilities that the role of Cdx4 in hematopoiesis is unique, or more likely, there is redundancy of the function within the Cdx family members.

Although Cdx4 may promote definitive HSCs formation and the surface antigens of Cdx4-expanded cells on OP9 displayed similar characters of AGM-HSCs, that is CD41+/CD31+/CD34+/c-kit+, and acquiring CD45+ along the differentiation, Cdx4 alone expanded cells did not engraft mice efficiently. Gene expression analysis showed that OP9 co-cultured cell expanded by HoxB4 induction (or retroviral transduction of HoxB4) have more than 100 fold increase of HoxB4 expression than cells expanded by Cdx4. If HoxB4 is the major factor in self-renewal and expansion of HSCs, weak enhancement of HoxB4 expression by Cdx4 may not be enough to maintain or expand transplantable HSCs on OP9. The majority of OP9 co-cultured cells may be consisted of multipotential or committed progenitors; and the long-term transplantable HSCs may only contribute to a very small percentage of the transplanted population. Nevertheless, the existence of lymphocytes and switching to β-major globin of OP9 co-cultured cells as well as low but long-term reconstitution after 8 weeks post transplantation demonstrated the existence of definitive HSCs in Cdx4-expanded cells.

The nature of the HSC as a self-renewing, multipotential blood progenitor was demonstrated definitively in the mid 1980s in experiments that coupled retroviral infection to bone marrow transplantation (Lemischka, 1986; Keller, 1985). Owing to semi-random integration of provirus within the genome of infected cells, retroviral transduction generates unique genetic markers that can be interrogated by Southern hybridization. The demonstration that highly purified lymphoid and myeloid blood cells in engrafted mice showed multiple common sites of proviral integration established that multiple blood lineages derived from single precursor cells. Some of these clones were again detected in the hematopoietic tissue of secondary recipient mice transplanted with the marrow from primary engrafted animals (Lemischka, 1986). The evidence that single clones can reconstitute the blood of both primary and secondary recipients demonstrated a long-lived set of precursors, and their detection in both lymphoid and myeloid lineages proved multi-lineage differentiation potential, thereby establishing the paradigmatic definition of stem cells as self-renewing multipotential progenitors. Although several groups demonstrated limited success of blood reconstitution of differentiated ESCs, no efforts endeavored in the clonal analysis on in vitro ESC-derived hematopoietic progenitors. In the present study, we have demonstrated ES-derived hematopoietic progenitors modified with Cdx4/HoxB4 were able to engraft lethally irradiated adult mice with long-term and multilineage blood reconstitution. Moreover, we apply the classical proviral integration analysis in engrafted blood lineages of primary and secondary mice to demonstrate the clonal derivation of HSCs from murine ESCs. The long-term hematopoietic reconstitution of primary and secondary mice with common clones demonstrates the self-renewal capacity of the ESC-derived hematopoietic precursors. Moreover, the evidence that myeloid and lymphoid cells derive from common clones demonstrates the multi-lineage differentiation potential of the ESC-derived cells. Taken together, our data validate the classical definition of a self-renewing, multi-lineage hematopoietic stem cell, and indicate the successful derivation of long-term HSCs from ESCs in vitro. Thus, we can achieve hematopoietic engraftment of irradiated mice by differentiating ESCs into the corresponding definitive adult HSC.

Comparing to other published studies, our system to derive transplantable HSCs is very efficient. Starting from 3×104 of ES cells, after in vitro EB differentiation and 10 to 14 days of OP9 co-culture, we could obtain 5-10×109 cells, which are enough to transplant 2500 to 5000 mice if using 2 million cells per mouse. This is 100 times more cells than we could obtain from whole bone morrow of a mouse (assume we can get 50 million bone morrow cells from one mouse).

### Example 2

### Derivation of Hematopoietic Stem Cells from Embryonic Stem Cells

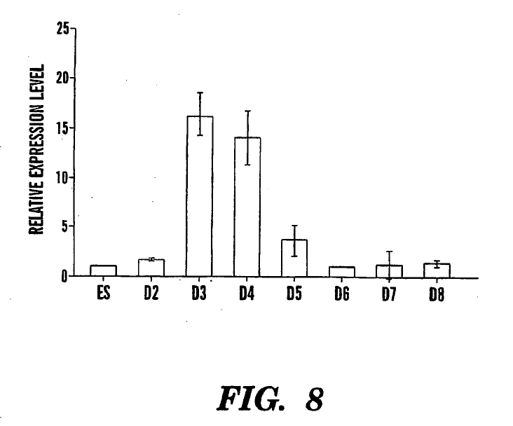

FIG. 8 shows quantitation of the results of an RT-PCR analysis of Cdx4 expression during embryoid body (EB) development. The data is shown as relative expression levels during different days.

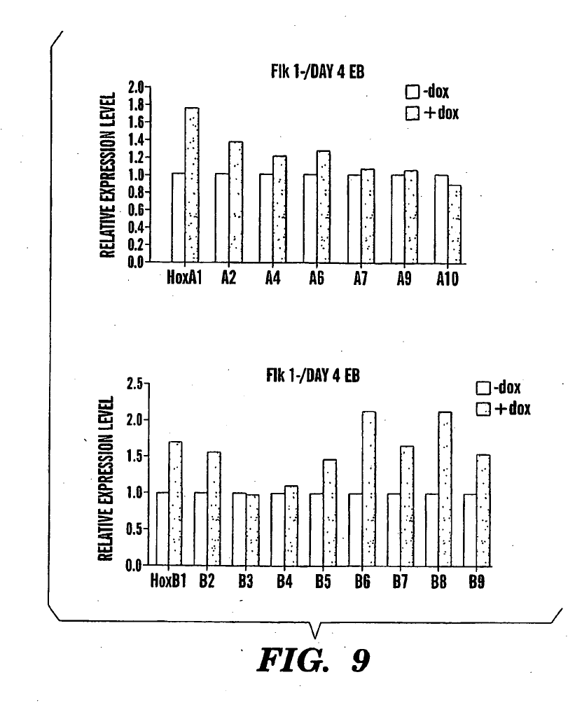

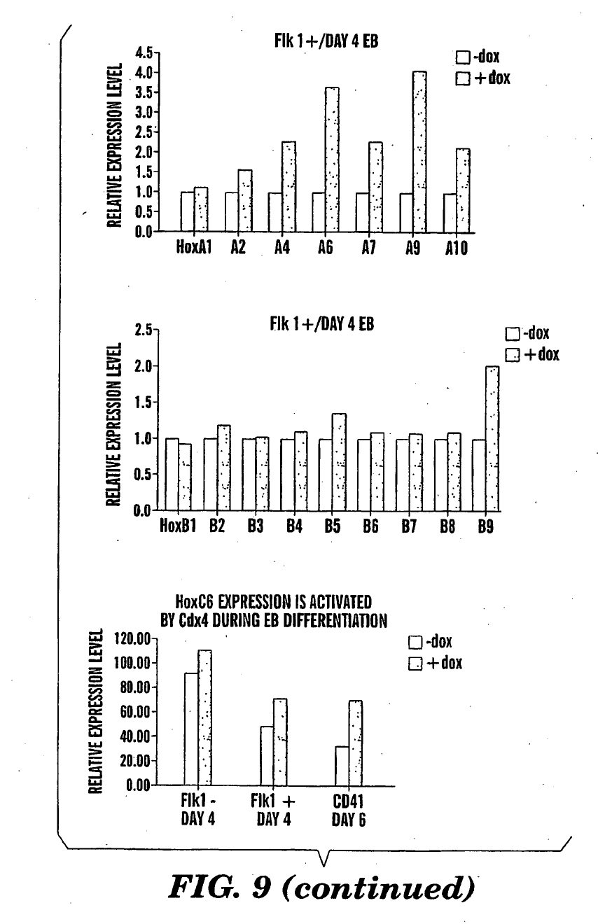

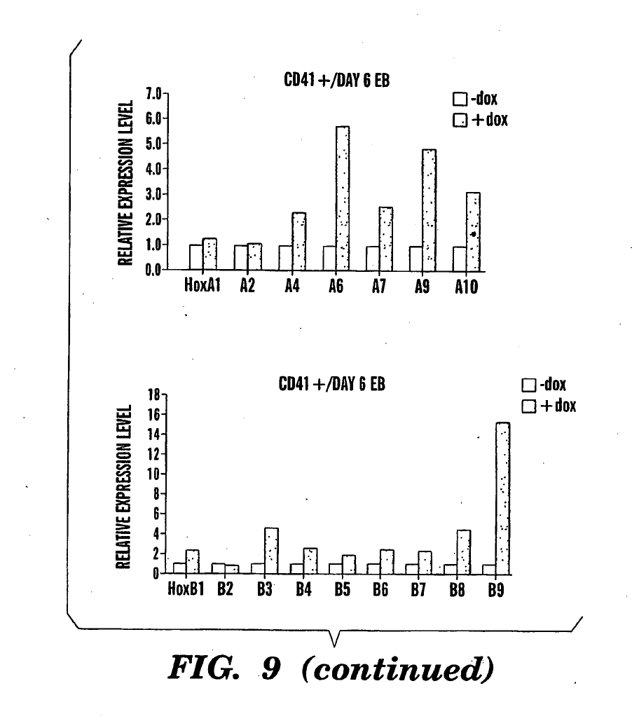

FIG. 9 shows that ectopic Cdx4 expression induces Hox gene expression in hematopoietic cells, including HoxA1, HoxA2, HoxA4, HoxA6, HoxA7, HoxA9, and HoxA10. Hematopoietic cells which are analyzed include Flk1-, day 4 EBs, Flk1-, day 4 EBs, and CD41+ day 6 EBs.

Protein transduction studies were done. See Wadia and Dowdy (Current Opinion in Biotechnology. 2002. 13:52-56) for a review methods that may be used to introduce proteins into a cell.

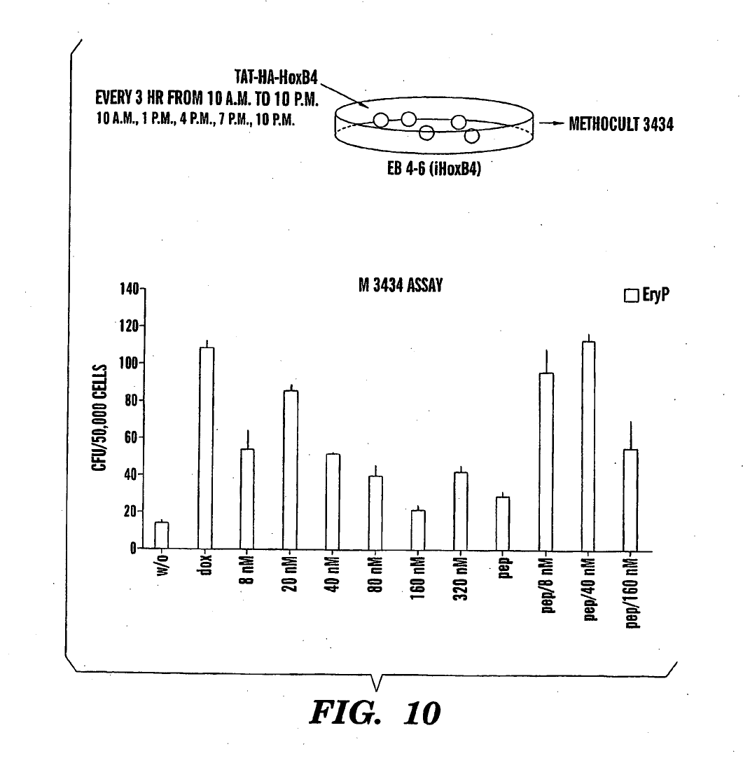

Proteins fused with a TAT-HA2 peptide enhance transduction into a cell (Wadia et al. 2004. Nat Med. 10:310-315). Thus, a plasmid was constructed for the generation of a TAT-HA-HoxB4 recombinant fusion protein. Anti-HA antibodies demonstrate the transduction of the fusion protein on 293 cells. The results of TAT-HA-HoxB4 fusion protein transduction on EBs are shown in FIG. 10.

In summary, we have shown that overexpression of Cdx4 promotes hemangioblast and hematopoietic progenitor formation; and enables ESC-derived hematopoietic progenitors to expand on OP9 stromal cells, undergo multilineage differentiation, and switch embryonic to adult type beta globin. Overexpression of Cdx4 also promotes the expression of certain hox genes. For example, expression of hoxa4, a6, a7, a9, and a10 was increased by cdx4 induction specifically in flk1+ and CD41+ cells.

Our results have also shown that the combination of cdx4/hoxb4 yields radioprotection; results in a high degree of multi-lineage donor chimerism; generates transplantable to secondary HSCs. Southern analysis of retroviral integration documents the clonality; and thus meets the gold standard of HSC function.

The references cited below and throughout the application are incorporated herein by reference.

### REFERENCES

1. Kyba, M. & Daley, G. Q. Hematopoiesis from embryonic stem cells: lessons from and for ontogeny. Exp Hematol 31, 994-1006 (2003).
2. Davidson, A. J. et al. Cdx4 mutants fail to specify blood progenitors and can be rescued by multiple hox genes. Nature 425, 300-6 (2003).
3. Lemischka, I. R., Raulet, D. H. & Mulligan, R. C. Developmental potential and dynamic behavior of hematopoietic stem cells. Cell 45, 917-27 (1986).
4. Keller, G., Paige, C., Gilboa, E. & Wagner, E. F. Expression of a foreign gene in myeloid and lymphoid cells derived from multipotent haematopoietic precursors. Nature 318, 149-54 (1985).
5. Sauvageau, G. et al. Overexpression of HOXB4 in hematopoietic cells causes the selective expansion of more primitive populations in vitro and in vivo. Genes Dev 9, 1753-65. (1995).
6. Kyba, M., Perlingeiro, R. C. & Daley, G. Q. HoxB4 confers definitive lymphoid-myeloid engraftment potential on embryonic stem cell and yolk sac hematopoietic progenitors. Cell 109, 29-37 (2002).
7. Rideout, W. M., 3rd, Hochedlinger, K., Kyba, M., Daley, G. Q. & Jaenisch, R. Correction of a genetic defect by nuclear transplantation and combined cell and gene therapy. Cell 109, 17-27. (2002).
8. Brun, A. C., Fan, X., Bjornsson, J. M., Humphries, R. K. & Karlsson, S. Enforced adenoviral vector-mediated expression of HOXB4 in human umbilical cord blood CD34+ cells promotes myeloid differentiation but not proliferation. Mol Ther 8, 618-28 (2003).
9. Schiedlmeier, B. et al. High-level ectopic HOXB4 expression confers a profound in vivo competitive growth advantage on human cord blood CD34+ cells, but impairs lymphomyeloid differentiation. Blood 101, 1759-68 (2003).
10. Mountford, P. et al. Dicistronic targeting constructs: reporters and modifiers of mammalian gene expression. Proc Natl Acad Sci USA 91, 4303-7 (1994).
Nakano, T., Kodama, H. & Honjo, T. Generation of lymphohematopoietic cells from embryonic stem cells in culture. Science 265, 1098-101 (1994).
12. Colucci, F. et al. Dissecting NK cell development using a novel alymphoid mouse model: investigating the role of the c-ab1 proto-oncogene in murine NK cell differentiation. J Immunol 162, 2761-5 (1999).
13. Jordan, C. T. & Lemischka, I. R. Clonal and systemic analysis of long-term hematopoiesis in the mouse. Genes Dev 4, 220-32 (1990).
14. Rideout, W. M., 3rd, Hochedlinger, K., Kyba, M., Daley, G. Q. & Jaenisch, R. Correction of a genetic defect by nuclear transplantation and combined cell and gene therapy. Cell 109, 17-27 (2002).
15. O'Gorman, S., Dagenais, N. A., Qian, M. & Marchuk, Y. Protamine-Cre recombinase transgenes efficiently recombine target sequences in the male germ line of mice, but not in embryonic stem cells. Proc Natl Acad Sci USA 94, 14602-7 (1997).
16. Livak, K. J. & Schmittgen, T. D. Analysis of relative gene expression data using real-time quantitative PCR and the 2(-Delta Delta C(T)) Method. Methods 25, 402-8 (2001).
17. Okabe, M., Ikawa, M., Kominami, K., Nakanishi, T. & Nishimune, Y. ‘Green mice’ as a source of ubiquitous green cells. FEBS Lett 407, 313-9 (1997).
18. Klug, C. A., Cheshier, S. & Weissman, I. L. Inactivation of a GFP retrovirus occurs at multiple levels in long-term repopulating stem cells and their differentiated progeny. Blood 96, 894-901 (2000).

We claim:

1. A method for inducing differentiation of an embryonic stem cell into a hematopoietic stem cell, comprising introducing into said stem cells in an in vitro culture medium an exogenous protein comprising at least one protein encoded by a gene selected from the group consisting of a cdx gene and a hox gene, and culturing said stem cells, thereby inducing its differentiation into a hematopoietic stem cell.

2. The method of claim 1, wherein the protein is introduced via an exogenous nucleic acid introduced into the stem cell, wherein each gene is operably linked to a promoter, and culturing said stem cells under conditions to express said gene(s) in the embryonic stem cell.

3. The method of claim 1, wherein the exogenous protein introduced into the stem cell is recombinant.

4. The method of claim 1, wherein the cdx gene is selected from the group consisting of cdx1, cdx2 and cdx4.

5. The method of claim 1, wherein the hox gene is selected from the group consisting of hoxa4, hoxa6, hoxa7, hoxa9, hoxa10, hoxb1, hoxb3, hoxb4, hoxb5, hoxb6, hoxb7, hoxb8, hoxb9 or hoxc6

6. The method of claim 1, wherein a cdx 4 gene and a hoxb4 gene are introduced into the stem cells.

7. The method of claim 1, wherein a cdx gene and a hox gene are introduced into the stem cells, and the cdx gene is expressed in the stem cells before the hox gene is expressed.

8. The method of claim 1, wherein the embryonic stem cell is a murine embryonic stem cell.

9. The method of claim 1, wherein the embryonic stem cell is a human embryonic stem cell.

10. A method for producing hematopoietic stem cells, comprising the steps of: (a) obtaining or generating a culture of embryonic stem cells; and (b) introducing into said stem cells in an in vitro culture medium an exogenous protein comprising protein encoded by at least one gene selected from the group consisting of a cdx gene and a hox gene, and culturing said stem cells, thereby producing hematopoietic stem cells

11. The method of claim 10, wherein the exogenous protein is introduced via introduction of nucleic acid into the stem cells, wherein each gene is operably linked to a promoter, and culturing said stem cells under conditions to express said gene(s) in the embryonic stem cell.

12. The method of claim 10, wherein the exogenous protein is full-length.

13. The method of claim 10, wherein the cdx gene is selected from the group consisting of cdx1, cdx2 and cdx4.

14. The method of claim 10, wherein the hox gene is selected from the group consisting of hoxa9, hoxb4, and hoxb7.

15. The method of claim 10, wherein a cdx 4 gene and a hoxb4 gene are introduced into the stem cells.

16. The method of claim 10, wherein a cdx gene and a hox gene are introduced into the stem cells, and the cdx gene is expressed in the stem cells before the hox gene is expressed.

17. The method of claim 10, wherein the embryonic stem cell is a murine embryonic stem cell.

18. The method of claim 10, wherein the embryonic stem cell is a human embryonic stem cell.

19. A method for enhancing proliferation or hematopoietic differentiation of a mammalian stem cell, comprising introducing into said stem cells in an in vitro culture medium an exogenous protein comprising protein encoded by at least one gene selected from the group consisting of a cdx gene and a hox gene, and culturing said stem cells, thereby enhancing proliferation or hematopoietic differentiation of a mammalian stem cell.

20. The method of claim 19, wherein the exogenous protein is introduced via introduction of a nucleic acid, wherein each gene is operably linked to a promoter, and culturing said stem cells under conditions to express said gene(s) in the embryonic stern cell.

21. The method of claim 19, wherein the exogenous protein is recombinant.

22. The method of claim 19, wherein the cdx is selected from the group consisting of cdx 1, 2 or 4.

23. The method of claim 19, wherein the hox is selected from the group consisting of hoxa9, hoxb4, or hoxb7.

24. The method of claim 19, wherein the stem cell is an embryonic stem cell.

25. The method of claim 19, wherein the stem cell is a hematopoietic stem cell.

26. The method of claim 19, wherein the cell is a CD34⁺ cell.

27. The method of claim 19, wherein the cell is autologous.

28. The method of claim 19, wherein the cell is obtained from a human.

29. The method of claim 28, wherein the human is suffering from, or is susceptible to, decreased blood cell levels.

30. The method of claim 29, wherein the decreased blood cell levels are caused by chemotherapy, radiation therapy, bone marrow transplantation therapy or congenital anemia.

31. The method of claim 19, wherein the exogenous nucleic acid is a retroviral vector.

32. The method of claim 19, wherein the exogenous nucleic acid is an episomal vector.

33. The method of claim 19, wherein the stem cell is an embryonic stem cell.

34. A method of treating a mammal in need of improved hematopoietic capability, comprising the steps of:
a) introducing into a stem cell an exogenous protein comprising protein encoded by at least one gene selected from the group consisting of a cdx and a hox gene;
b) culturing said stem cells, thereby enhancing proliferation or hematopoietic differentiation of the stem cells; and
c) administering said cells to the mammal, thereby hematopoietic capability is improved.

35. The method of claim 34, wherein the exogenous protein is introduced via introduction of an exogenous nucleic acid and the stem cells are cultured under conditions to express said gene(s) in the stem cell.

36. The method of claim 34, wherein the exogenous protein is recombinant.

37. The method of claim 34, wherein the cdx is selected from the group consisting of cdx 1, 2 or 4.

38. The method of claim 34, wherein the hox is selected from the group consisting of hoxa4, hoxa6, hoxa7, hoxa9, hoxa10, hoxb1, hoxb3, hoxb4, hoxb5, hoxb6, hoxb7, hoxb8, hoxb9 or hoxc6.

39. The method of claim 34, wherein the cdx gene is expressed before the hox gene is expressed.

40. The method of claim 34, wherein the mammal is a human.

41. The method of claim 34, wherein the cell is autologous.

42. The method of claim 34, wherein the human is suffering from, or is susceptible to, decrease blood cell levels.

43. The method of claim 42, wherein the decreased blood cell levels are caused by chemotherapy, radiation therapy, bone marrow transplantation therapy, or congenital anemia.

44. A method for inducing differentiation of a stem cell into a hematopoietic stem cell, comprising introducing into said stem cells in an in vitro culture medium an exogenous protein comprising a protein encoded by at least one gene selected from the group consisting of a cdx gene and a hox gene, and culturing said stem cells, thereby inducing its differentiation into a hematopoietic stem cell.

45. The method of claim 44, wherein the exogenous protein is introduced via introduction of an exogenous nucleic acid and the stem cells are cultured under conditions to express said gene(s) in the stem cell.

46. The method claim 44, wherein the exogenous protein is recombinant.

47. The method of claim 44, wherein the stem cell is selected from the group consisting of embryonic stem cells, umbilical cord blood stem cells, unrestricted somatic stem cells (USSC) derived from human umbilical cord blood, placenta-derived stem cells, somatic stem cells, mesenchymal stem cells, mesenchymal progenitor cells, hematopoietic stem cells, hematopoietic lineage progenitor cells, endothelial stem cells, placental fetal stem cells and endothelial progenitor cells.

48. The method of claim 44, wherein the cdx gene is selected from the group consisting of cdx1, cdx2 and cdx4.

49. The method of claim 44, wherein the hox gene is selected from the group consisting of hoxa9, hoxb4, and hoxb7.

50. The method of claim 44, wherein a cdx 4 gene and a hoxb4 gene are introduced into the stem cells.

51. The method of claim 44, wherein a cdx gene and a hox gene are introduced into the stem cells, and the cdx gene is expressed in the stem cells before the hox gene is expressed.

52. The method of claim 1, 10, 19, 34, 44, wherein the exogenous protein is introduced via cells in culture with the stem cells, wherein the cells in culture with the stem cells produce the exogenous protein.
